(19) United States
(12) Patent Application Publication
(10) Pub. No.: US 2023/0148984 A1
(43) Pub. Date: May 18, 2023

(54) SYSTEMS AND METHODS FOR RADIATION DOSE MANAGEMENT

(71) Applicant: SHANGHAI UNITED IMAGING HEALTHCARE CO., LTD., Shanghai (CN)

(72) Inventor: Yan WANG, Shanghai (CN)

(73) Assignee: SHANGHAI UNITED IMAGING HEALTHCARE CO., LTD., Shanghai (CN)

(21) Appl. No.: 18/157,779

(22) Filed: Jan. 20, 2023

Related U.S. Application Data
(63) Continuation of application No. PCT/CN2022/076696, filed on Feb. 17, 2022.

(30) Foreign Application Priority Data
Feb. 24, 2021 (CN) 202110206504.9

(51) Int. Cl. A61B 6/00; A61B 6/02; A61B 6/03

(52) U.S. Cl. CPC A61B 6/542; A61B 6/027; A61B 6/032

(57) ABSTRACT

The present disclosure is related to systems and methods for radiation dose management. The method includes obtaining scan information of a subject. The scan information includes a scan mode, a scan region of the subject, and a radiation dose of the subject. The method includes determining a record mode based on the scan mode. The method includes recording the scan region and the radiation dose based on the record mode.

### CROSS-REFERENCE TO RELATED APPLICATIONS

This application is a Continuation of International Application No. PCT/CN2022/076696, filed on Feb. 17, 2022, which claims priority of Chinese Patent Application No. 202110206504.9, filed on Feb. 24, 2021, and the contents of each of which are hereby incorporated by reference.

### TECHNICAL FIELD

This disclosure generally relates to systems and methods for medical imaging, and more particularly, relates to systems and methods for radiation dose management.

### BACKGROUND

Medical systems, such as a CT device, an MRI device, a PET device, are widely used for generating images of the interior of a patient for medical diagnosis and/or treatment purposes. After a scan (e.g., a CT scan) is performed on a patient, it is often necessary to determine a radiation dose distribution in the body of the patient to ensure that a tumor area receives a planning radiation dose, and/or that the radiation dose that a normal tissue receives is within a safe range. In addition, a patient may receive a plurality of scans in a time period (e.g., one week, one month), a total radiation dose distribution in the body of the patient needs to be determined in order to avoid the accumulation of excessive radiation dose in the patient. The reasonable determination of a scan plan of the patient relies on accurate management and presentation of historical radiation dose distributions of the patient. Thus, it is desired to provide systems and methods for managing a radiation dose of a subject accurately and efficiently.

### SUMMARY

According to an aspect of the present disclosure, a method for radiation dose management may be implemented on a computing device having one or more processors and one or more storage devices. The method may include obtaining scan information of a subject. The scan information may include a scan mode, a scan region of the subject, and a radiation dose of the subject. The method may include determining a record mode based on the scan mode. The method may include recording the scan region and the radiation dose based on the record mode.

In some embodiments, the method may include determining a radiation dose corresponding to each position of a plurality of positions distributed along at least one direction of the scan region based on the radiation dose of the subject. The method may include recording the radiation dose corresponding to the each position of the plurality of positions of the scan region based on the record mode.

In some embodiments, the scan mode may include a spiral CT scan. The method may include determining the record mode as a radiation dose curve based on the spiral CT scan.

In some embodiments, the method may include determining a radiation dose of each layer of a plurality of layers of the scan region. The method may include determining the radiation dose curve based on radiation doses of the plurality of layers of the scan region. The method may include recording the radiation doses corresponding to the plurality of layers of the scan region using the radiation dose curve.

In some embodiments, the scan mode may include a CT scan. The method may include determining the record mode as a first model including a plurality of elliptical cylinders based on the CT scan. The first model may be configured to reflect radiation doses corresponding to a plurality of layers of the scan region.

In some embodiments, the scan information may further include a thickness of each layer of the plurality of layers of the scan region. The method may include determining a radiation dose of the each layer of the plurality of layers of the scan region. The method may include generating the first model based on the thickness of the each layer of the plurality of layers of the scan region, and the radiation dose of the each layer of the plurality of layers of the scan region. A height of each elliptical cylinder of the plurality of elliptical cylinders may correspond to a thickness of a corresponding layer of the plurality of layers of the scan region. A cross-sectional area of the each elliptical cylinder may correspond to a radiation dose of the corresponding layer. The method may include recording radiation doses corresponding to the plurality of layers of the scan region using the first model.

In some embodiments, the scan mode may include a CT scan. The scan region may include a reference region. The method may include determining the record mode as a second model based on the CT scan and the reference region. The second model may include a cylinder with a first curved surface. The first curved surface may correspond to the reference region.

In some embodiments, the scan information may further include a radiation dose of the reference region. The method may include generating the cylinder based on the scan region and the radiation dose of the subject. A longitudinal axis of the cylinder may be perpendicular to a cross section of the subject. The method may include generating the second model by forming, based on the radiation dose of the reference region, the first curved surface on the cylinder. The method may include recording radiation doses corresponding to a plurality of positions of the reference region using the second model.

In some embodiments, the scan mode may include a CT scan. The scan region may include a reference region. The method may include determining the record mode as a third model based on the CT scan and the reference region. The third model may include a plurality of elliptical cylinders with a second curved surface. The second curved surface may correspond to the reference region. The third model may be configured to reflect radiation doses corresponding to a plurality of layers of the scan region.

In some embodiments, the scan information may further include a thickness of each layer of the plurality of layers of the scan region, and a radiation dose of the reference region. The method may include determining a radiation dose of the each layer of the plurality of layers of the scan region. The method may include generating the plurality of elliptical cylinders based on the thickness of the each layer of the plurality of layers of the scan region, and the radiation dose of the each layer of the plurality of layers of the scan region. A longitudinal axis of the each elliptical cylinder of the plurality of elliptical cylinders may be located on a same straight line. The longitudinal axis of the each elliptical cylinder of the plurality of elliptical cylinders may be perpendicular to a cross section of the subject. A height of the each elliptical cylinder of the plurality of elliptical cylinders may correspond to a thickness of a corresponding layer of the plurality of layers of the scan region. A cross-sectional area of the each elliptical cylinder may correspond to a radiation dose of the corresponding layer. The method may include generating the third model by forming, based on the radiation dose of the reference region, the second curved surface on the plurality of elliptical cylinders. The method may include recording radiation doses corresponding to the plurality of layers of the scan region and a plurality of positions of the reference region using the third model.

In some embodiments, the scan mode may include a beam limited scan mode. The method may include determining the record mode as a fourth model based on the beam limited scan mode. The fourth model may include a frustum.

In some embodiments, the scan information may further include a shape of a beam limiter of a medical device. The method may include generating the fourth model based on the shape of the beam limiter and the radiation dose of the subject. An axis of the frustum may be perpendicular to a coronal plane of the subject. A shape of a surface of the frustum may correspond to the shape of the beam limiter. A height of the frustum may correspond to the radiation dose of the subject. The method may include recording the radiation dose of the subject using the fourth model.

In some embodiments, the method may include obtaining one or more historical scan regions of the subject, one or more historical radiation doses of the subject, and one or more historical record modes. The method may include generating a subject model representing the subject. The method may include causing a terminal device to display the subject model, the one or more historical scan regions and the one or more historical radiation doses according to the one or more historical record modes.

In some embodiments, the method may include generating target image data by mapping the one or more historical scan regions and the one or more historical radiation doses on the subject model according to the one or more historical record modes. The method may include causing the terminal device to display the target image data.

In some embodiments, the one or more historical scan regions may be displayed in the subject model in different colors. One or more colors of the one or more historical scan regions may correspond to the one or more historical radiation doses of the one or more historical scan regions.

In some embodiments, the method may include generating a model based on the record mode. The method may include visualizing the scan region and the radiation dose using the model.

In some embodiments, the scan information may include historical scan information of at least one historical scan of the subject. The method may include displaying a current distribution of an accumulative radiation dose corresponding to the at least one historical scan of the subject in the model.

In some embodiments, the scan information may further include current scan information of a current scan of the subject. The method may include displaying an estimated distribution of an accumulative radiation dose corresponding to the at least one historical scan of the subject and the current scan of the subject in the model.

According to an aspect of the present disclosure, a method for radiation dose management may be implemented on a computing device having one or more processors and one or more storage devices. The method may include obtaining scan information of a subject. The method may include determining, based on the scan information, a distribution of a radiation dose in at least one dimension of a scan region of the subject. The method may include displaying the distribution of the radiation dose in a visualization model of at least a portion of the subject.

According to an aspect of the present disclosure, a method for radiation dose management may be implemented on a computing device having one or more processors and one or more storage devices. The method may include obtaining historical scan information of a plurality of historical scans of a subject. The method may include determining, based on the historical scan information, a current distribution of an accumulative radiation dose corresponding to the plurality of historical scans in at least one dimension of the subject. The method may include displaying the current distribution of the accumulative radiation dose in a visualization model of the subject.

In some embodiments, the historical scan information of each historical scan of the plurality of historical scans of the subject may include a historical scan region of the subject, and a historical radiation dose of the subject in the historical scan. The method may include determining a time weight corresponding to the each historical scan of the plurality of historical scans based on a time of the each historical scan. The method may include determining the current distribution of the accumulative radiation dose corresponding to the plurality of historical scans based on the historical radiation dose of the subject in the each historical scan of the plurality of historical scans and the time weight corresponding to the each historical scan of the plurality of historical scans.

In some embodiments, the historical scan information of each historical scan of the plurality of historical scans of the subject may include a historical scan mode, a historical scan region of the subject, and a historical radiation dose of the subject in the scan. The method may include determining a record mode based on the historical scan mode of the each scan of the plurality of scans of the subject. The method may include generating the visualization model based on the record mode. The method may include displaying the current distribution of the accumulative radiation dose in the visualization model.

According to another aspect of the present disclosure, a system may include at least one storage device storing a set of instructions, and at least one processor in communication with the at least one storage device. When executing the stored set of instructions, the at least one processor may cause the system to perform a method. The method may include obtaining scan information of a subject. The scan information may include a scan mode, a scan region of the subject, and a radiation dose of the subject. The method may include determining a record mode based on the scan mode. The method may include recording the scan region and the radiation dose based on the record mode.

According to another aspect of the present disclosure, a system may include at least one storage device storing a set of instructions, and at least one processor in communication with the at least one storage device. When executing the stored set of instructions, the at least one processor may cause the system to perform a method. The method may include obtaining scan information of a subject. The method may include determining, based on the scan information, a distribution of a radiation dose in at least one dimension of a scan region of the subject. The method may include displaying the distribution of the radiation dose in a visualization model of at least a portion of the subject.

According to another aspect of the present disclosure, a system may include at least one storage device storing a set of instructions, and at least one processor in communication with the at least one storage device. When executing the stored set of instructions, the at least one processor may cause the system to perform a method. The method may include obtaining historical scan information of a plurality of historical scans of a subject. The method may include determining, based on the historical scan information, a current distribution of an accumulative radiation dose corresponding to the plurality of historical scans in at least one dimension of the subject. The method may include displaying the current distribution of the accumulative radiation dose in a visualization model of the subject.

According to another aspect of the present disclosure, a non-transitory computer readable medium may include at least one set of instructions. When executed by at least one processor of a computing device, the at least one set of instructions may cause the at least one processor to effectuate a method. The method may include obtaining scan information of a subject. The scan information may include a scan mode, a scan region of the subject, and a radiation dose of the subject. The method may include determining a record mode based on the scan mode. The method may include recording the scan region and the radiation dose based on the record mode.

According to another aspect of the present disclosure, a non-transitory computer readable medium may include at least one set of instructions. When executed by at least one processor of a computing device, the at least one set of instructions may cause the at least one processor to effectuate a method. The method may include obtaining scan information of a subject. The method may include determining, based on the scan information, a distribution of a radiation dose in at least one dimension of a scan region of the subject. The method may include displaying the distribution of the radiation dose in a visualization model of at least a portion of the subject.

According to another aspect of the present disclosure, a non-transitory computer readable medium may include at least one set of instructions. When executed by at least one processor of a computing device, the at least one set of instructions may cause the at least one processor to effectuate a method. The method may include obtaining historical scan information of a plurality of historical scans of a subject. The method may include determining, based on the historical scan information, a current distribution of an accumulative radiation dose corresponding to the plurality of historical scans in at least one dimension of the subject. The method may include displaying the current distribution of the accumulative radiation dose in a visualization model of the subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device on which a processing device may be implemented according to some embodiments of the present disclosure;

FIG. 3 is a schematic diagram illustrating exemplary hardware and/or software components of an exemplary mobile device according to some embodiments of the present disclosure;

FIG. 4 is a schematic diagram illustrating an exemplary processing device according to some embodiments of the present disclosure;

FIG. 5 is a flowchart illustrating an exemplary process for recording a scan region and a radiation dose based on a record mode according to some embodiments of the present disclosure;

FIG. 6 is a flowchart illustrating an exemplary process for displaying a subject model, one or more historical scan regions, and one or more historical radiation doses according to one or more historical record modes according to some embodiments of the present disclosure;

FIG. 7 is a schematic diagram illustrating an exemplary radiation dose curve according to some embodiments of the present disclosure;

FIG. 8 is a schematic diagram illustrating an exemplary first model according to some embodiments of the present disclosure;

FIG. 9 is a schematic diagram illustrating an exemplary elliptical cylinder according to some embodiments of the present disclosure;

FIG. 10 is a schematic diagram illustrating an exemplary second model according to some embodiments of the present disclosure;

FIG. 11 is a schematic diagram illustrating an exemplary third model according to some embodiments of the present disclosure;

FIG. 12 is a schematic diagram illustrating an exemplary fourth model according to some embodiments of the present disclosure;

FIG. 13 is a schematic diagram illustrating exemplary target image data according to some embodiments of the present disclosure;

FIGS. 14A-14C are schematic diagrams illustrating exemplary historical scan processes of a subject according to some embodiments of the present disclosure.

FIG. 15A is a schematic diagram illustrating an exemplary user interface according to some embodiments of the present disclosure;

FIG. 15B is a schematic diagram illustrating an exemplary user interface according to some embodiments of the present disclosure;

FIG. 16 is a schematic diagram illustrating an exemplary user interface according to some embodiments of the present disclosure;

FIG. 17 is a schematic diagram illustrating an exemplary user interface according to some embodiments of the present disclosure;

FIG. 18 is a schematic diagram illustrating an exemplary user interface according to some embodiments of the present disclosure;

FIG. 19 is a schematic diagram illustrating an exemplary user interface according to some embodiments of the present disclosure;

FIG. 20 is a schematic diagram illustrating an exemplary user interface according to some embodiments of the present disclosure;

FIGS. 21A-21C are schematic diagrams illustrating exemplary scan processes of a subject according to some embodiments of the present disclosure; and

FIGS. 22A-22C are schematic diagrams illustrating exemplary scan processes of a subject according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

An aspect of the present disclosure relates to a system and method for radiation dose management. According to some embodiments of the present disclosure, a processing device may obtain scan information of a subject. The scan information may include a scan mode, a scan region of the subject, and a radiation dose of the scan region. The processing device may determine a record mode based on the scan mode. The processing device may record the scan region and the radiation dose based on the record mode. In some embodiments, the processing device may generate a subject model representing the subject. The processing device may cause a terminal device to display the subject model, the scan region, and the radiation dose according to the record mode.

Accordingly, a distribution of the radiation dose in at least one dimension of the scan region of the subject during a scan may be displayed. A user (e.g., a doctor) of a medical device may view the distribution of the radiation dose of the subject intuitively, and/or manage the distribution of the radiation dose of the subject effectively. In addition, the processing device may obtain one or more historical scan regions of the subject, one or more historical radiation doses of the subject, and one or more historical record modes. The processing device may cause the terminal device to display the subject model, the one or more historical scan regions, and the one or more historical radiation doses according to the one or more historical record modes. Accordingly, a distribution of an accumulative radiation dose corresponding to a plurality of historical scans in at least one dimension of the subject may be displayed. The user (e.g., a doctor) of the medical device may determine and/or adjust a scan plan of the subject in a current scan based on the distribution of the accumulative radiation dose corresponding to the plurality of historical scans of the subject, which may prevent the subject (or a portion thereof) from receiving excessive radiation, and improve the safety of the subject during the current scan.

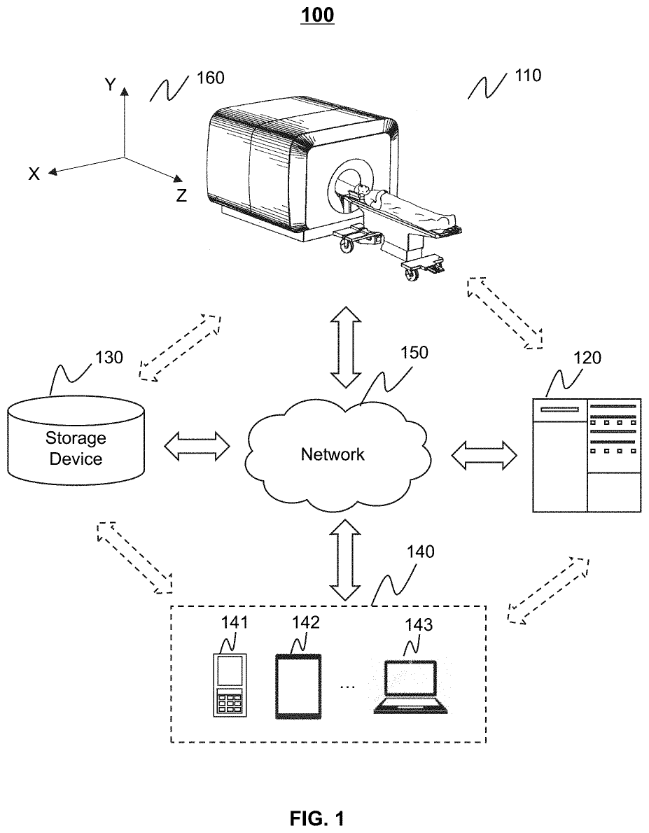

FIG. 1 is a schematic diagram illustrating an exemplary medical system according to some embodiments of the present disclosure. As illustrated, a medical system 100 may include a medical device 110, a processing device 120, a storage device 130, a terminal 140, and a network 150. The components of the medical system100 may be connected in one or more of various ways. Merely by way of example, as illustrated in FIG. 1, the medical device 110 may be connected to the processing device 120 directly as indicated by the bi-directional arrow in dotted lines linking the medical device 110 and the processing device 120, or through the network 150. As another example, the storage device 130 may be connected to the medical device 110 directly as indicated by the bi-directional arrow in dotted lines linking the medical device 110 and the storage device 130, or through the network 150. As still another example, the terminal 140 may be connected to the processing device 120 directly as indicated by the bi-directional arrow in dotted lines linking the terminal 140 and the processing device 120, or through the network 150.

The medical device 110 may be configured to acquire imaging data relating to a subject. The imaging data relating to a subject may include an image (e.g., an image slice), projection data, or a combination thereof. In some embodiments, the imaging data may be a two-dimensional (2D) imaging data, a three-dimensional (3D) imaging data, a four-dimensional (4D) imaging data, or the like, or any combination thereof. The subject may be biological or non-biological. For example, the subject may include a patient, a man-made object, etc. As another example, the subject may include a specific portion, an organ, and/or tissue of the patient. Specifically, the subject may include the head, the neck, the thorax, the heart, the stomach, a blood vessel, soft tissue, a tumor, or the like, or any combination thereof. In the present disclosure, “object” and “subject” are used interchangeably.

In some embodiments, the medical device 110 may include a single modality imaging device. For example, the medical device 110 may include a positron emission tomography (PET) device, a single-photon emission computed tomography (SPECT) device, a magnetic resonance imaging (MRI) device (also referred to as an MR device, an MR scanner), a computed tomography (CT) device, an ultrasound (US) device, an X-ray imaging device, or the like, or any combination thereof. In some embodiments, the medical device 110 may include a multi-modality imaging device. Exemplary multi-modality imaging devices may include a PET-CT device, a PET-MRI device, a SPET-CT device, or the like, or any combination thereof. The multi-modality imaging device may perform multi-modality imaging simultaneously. For example, the PET-CT device may generate structural X-ray CT data and functional PET data simultaneously in a single scan. The PET-MRI device may generate MRI data and PET data simultaneously in a single scan.

The processing device 120 may process data and/or information obtained from the medical device 110, the storage device 130, and/or the terminal(s) 140. For example, the processing device 120 may obtain scan information of a subject. As another example, the processing device 120 may determine a record mode based on a scan mode. As another example, the processing device 120 may record a scan region and a radiation dose based on a record mode. In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, and/or the terminal(s) 140 via the network 150. As another example, the processing device 120 may be directly connected to the medical device 110, the terminal(s) 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal 140. In some embodiments, the processing device 120 may be part of the medical device 110.

The storage device 130 may store data, instructions, and/or any other information. In some embodiments, the storage device 130 may store data obtained from the medical device 110, the processing device 120, and/or the terminal(s) 140. The data may include image data acquired by the processing device 120, algorithms and/or models for processing the image data, etc. For example, the storage device 130 may store scan information of a subject. As another example, the storage device 130 may store record mode determined by the processing device 120. As another example, the storage device 130 may store a subject model determined by the processing device 120. As another example, the storage device 130 may store a distribution of a radiation dose in at least one dimension of a scan region of a subject determined by the processing device 120. As another example, the storage device 130 may store a distribution of an accumulative radiation dose corresponding to a plurality of scans in at least one dimension of a subject determined by the processing device 120. In some embodiments, the storage device 130 may store data and/or instructions that the processing device 120 and/or the terminal 140 may execute or use to perform exemplary methods described in the present disclosure. In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), a high-speed RAM, etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components in the medical system100 (e.g., the processing device 120, the terminal(s) 140). One or more components in the medical system100 may access the data or instructions stored in the storage device 130 via the network 150. In some embodiments, the storage device 130 may be integrated into the medical device 110.

The terminal(s) 140 may be connected to and/or communicate with the medical device 110, the processing device 120, and/or the storage device 130. In some embodiments, the terminal 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

In some embodiments, a hospital information system (HIS), a laboratory information management system (LIS), a picture archiving and communication systems (PACS), a radioiogy information system (RIS), or the like, or any combinaiton thereof, may be implemented on the terminal(s) 140.

The network 150 may include any suitable network that can facilitate the exchange of information and/or data for the medical system 100. In some embodiments, one or more components of the medical system 100 (e.g., the medical device 110, the processing device 120, the storage device 130, the terminal(s) 140, etc.) may communicate information and/or data with one or more other components of the medical system 100 via the network 150. For example, the processing device 120 and/or the terminal 140 may obtain scan information of a subject from the medical device 110 via the network 150. As another example, the processing device 120 and/or the terminal 140 may obtain information stored in the storage device 130 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, witches, server computers, and/or any combination thereof. For example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the medical system100 may be connected to the network 150 to exchange data and/or information.

In some embodiments, a medical coordinate system 160 (also referred to as a first coordinate system associated with a medical device) may be provided for the medical system 100 to define a position of a component (e.g., an absolute position, a position relative to another component) and/or a movement of the component. For illustration purposes, the medical coordinate system 160 may include the X-axis, the Y-axis, and the Z-axis. The X-axis and the Z-axis shown in FIG. 1 may be horizontal, and the Y-axis may be vertical. As illustrated, a positive X direction along the X-axis may be from the right side to the left side of a scanning table viewed from the direction facing the front of the medical device 110; a positive Y direction along the Y-axis may be from the lower part (or from the floor where the medical device 110 stands) to the upper part of a gantry of the medical device 110; and a positive Z direction along the Z-axis may be the direction in which the scanning table is moved out of a scanning channel (or referred to as a bore) of the medical device 110 viewed from the direction facing the front of the medical device 110.

It should be noted that the provided medical coordinate system 160 is illustrative, and not intended to limit the scope of the present disclosure. For example, the medical coordinate system 160 may only include two axes (e.g., the X-axis and the Y-axis). In addition, although the following descriptions discuss through various examples to determine a position of an entity by determining a coordinate of an entity in a certain coordinate system, it should be understood that the position of the entity may be determined by determining a coordinate of the entity in another coordinate system (e.g., a coordinate system that has a known transformation relationship with the certain medical coordinate system). For the convenience of descriptions, coordinates of an entity along an X-axis, a Y-axis, and a Z-axis in a coordinate system are also referred to as an X-coordinate, a Y-coordinate, and a Z-coordinate of the entity in the coordinate system, respectively.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure.

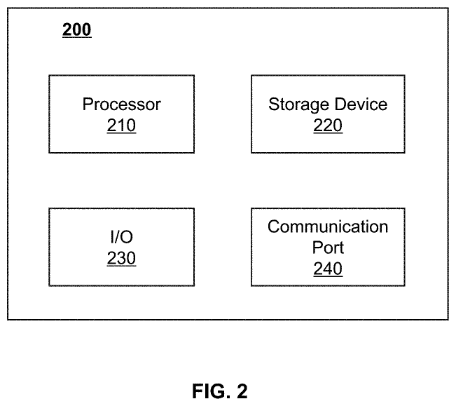

FIG. 2 is a schematic diagram illustrating exemplary hardware and/or software components of an exemplary computing device on which the processing device 120 may be implemented according to some embodiments of the present disclosure. As illustrated in FIG. 2, a computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process image data obtained from the medical device 110, the terminal device 140, the storage device 130, and/or any other component of the medical system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage device 220 may store data/information obtained from the medical device 110, the terminal device 140, the storage device 130, and/or any other component of the medical system 100. The storage device 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the terminal device 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

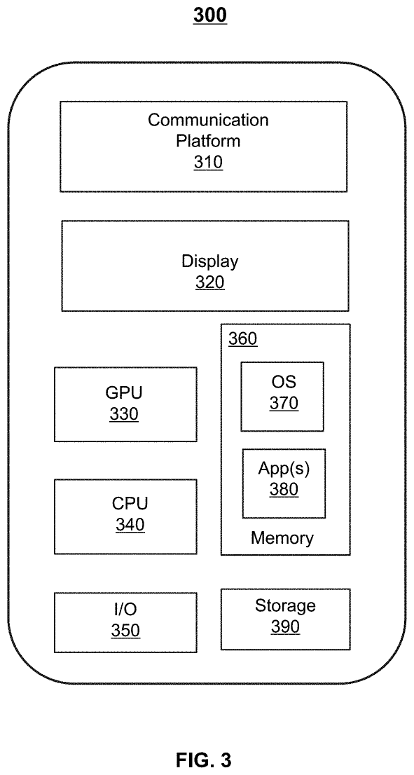

FIG. 3 is a schematic diagram illustrating exemplary hardware and/or software components of an exemplary mobile device according to some embodiments of the present disclosure. In some embodiments, the terminal device 140 and/or the processing device 120 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the medical system 100, and enable data and/or signal to be transmitted between the mobile device 300 and other components of the medical system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the medical system 100. For example, the communication platform 310 may transmit data and/or signals inputted by a user to other components of the medical system 100. The inputted data and/or signals may include a user instruction. As another example, the communication platform 310 may receive data and/or signals transmitted from the processing device 120. The received data and/or signals may include imaging data acquired by the medical device 110.

In some embodiments, a mobile operating system (OS) 370 (e.g., iOS™ Android™, Windows Phone™, etc.) and one or more applications (App(s)) 380 may be loaded into the memory 360 from the storage 390 in order to be executed by the CPU 340. The applications 380 may include a browser or any other suitable mobile apps for receiving and rendering information from the processing device 120. User interactions with the information stream may be achieved via the I/O 350 and provided to the processing device 120 and/or other components of the medical system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

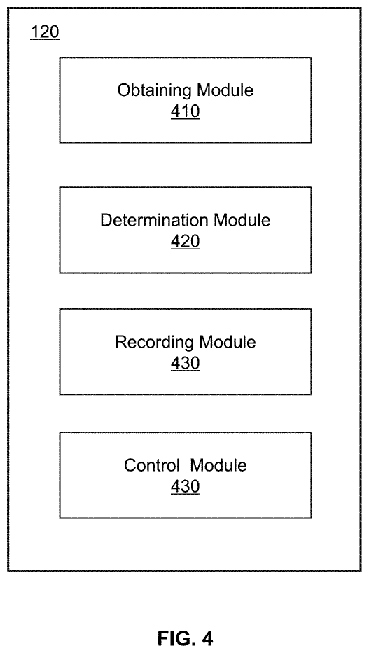

FIG. 4 is a schematic diagram illustrating an exemplary processing device according to some embodiments of the present disclosure. In some embodiments, the processing device 120 may include an obtaining module 410, a determination module 420, a recording module 430, and a control module 440.

The obtaining module 410 may be configured to obtain data and/or information associated with the medical system 100. The data and/or information associated with the medical system 100 may include scan information of a subject, a scan mode, a scan region of a subject, a radiation dose of a scan region, or the like, or any combination thereof. For example, the obtaining module 410 may obtain scan information of a subject. As another example, the obtaining module 410 may obtain one or more historical scan regions of a subject, one or more historical radiation doses of the subject, and one or more historical record modes. In some embodiments, the obtaining module 410 may obtain the data and/or the information associated with the medical system 100 from one or more components (e.g., the medical device 110, the storage device 130, the terminal 140) of the medical system 100 via the network 150.

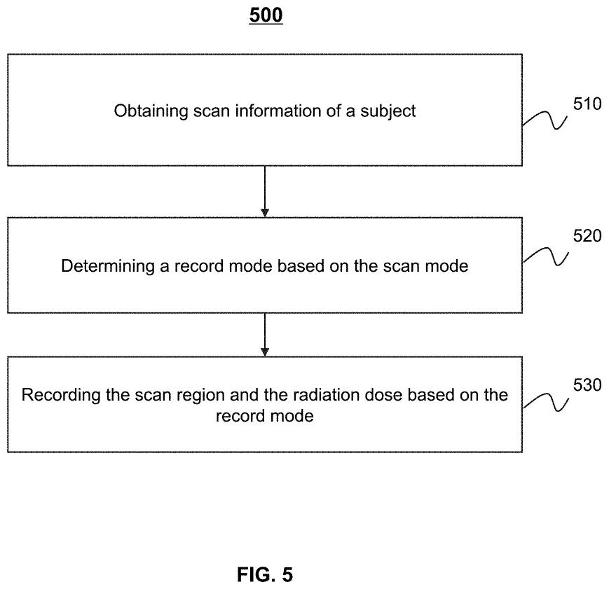

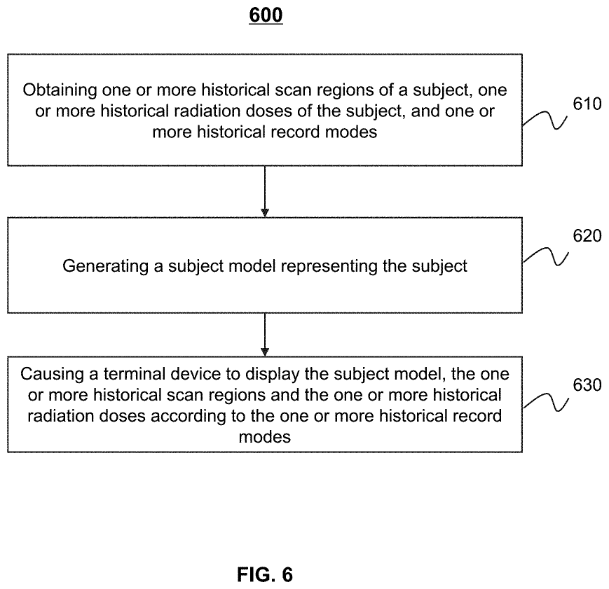

The determination module 420 may be configured to determine data and/or information associated with the medical system 100. In some embodiments, the determination module 420 may determine a record mode based on a scan mode. For example, the determination module 420 may determine a record mode based on a scan mode and a relationship between the record mode and the scan mode. More descriptions for determining a record mode may be found elsewhere in the present disclosure (e.g., FIG. 5 and descriptions thereof). For example, the determination module 420 may generate a subject model representing the subject. More descriptions for generating a subject model may be found elsewhere in the present disclosure (e.g., FIG. 6 and descriptions thereof).

The recording module 430 may be configured to record a scan region and a radiation dose based on a record mode. For example, the recording module 430 may record radiation doses corresponding to a plurality of layers of a scan region using a radiation dose curve. As another example, the recording module 430 may record radiation doses corresponding to a plurality of layers of a scan region using the first model including a plurality of elliptical cylinders. As still another example, the recording module 430 may record radiation doses corresponding to a plurality of positions of a reference region using a second model including a cylinder with a first curved surface. As still another example, the recording module 430 may record radiation doses corresponding to a plurality of layers of a scan region and a plurality of positions of a reference region using a third model including a plurality of elliptical cylinders with a second curved surface. As still another example, the recording module 430 may record radiation dose of a scan region using a fourth model including a frustum. More descriptions for recording a scan region and a radiation dose based on a record mode may be found elsewhere in the present disclosure (e.g., FIG. 5 and descriptions thereof).

The control module 440 may be configured to control one or more components (e.g., the medical device 110) of the medical system 100. For example, the control module 430 cause a terminal device (e.g., the terminal device 140) to display a subject model, one or more historical scan regions and one or more historical radiation doses according to one or more historical record modes. More descriptions for displaying a subject model, one or more historical scan regions and one or more historical radiation doses according to one or more historical record modes may be found elsewhere in the present disclosure (e.g., FIG. 6 and descriptions thereof).

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the determination module 420 and the recording module 430 may be combined into a single module. In some embodiments, one or more modules may be added or omitted in the processing device 120. For example, the processing device 120 may further include a storage module (not shown in FIG. 4) configured to store data and/or information (e.g., scan information, a record mode, a subject model) associated with the medical system 100.

FIG. 5 is a flowchart illustrating an exemplary process for recording a scan region and a radiation dose based on a record mode according to some embodiments of the present disclosure. In some embodiments, process 500 may be executed by the medical system 100. For example, the process 500 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the obtaining module 410) may obtain scan information of a subject.

In some embodiments, the subject may include a biological subject and/or a non-biological subject. For example, the subject may include a specific portion of a body, such as the head, the thorax, the abdomen, or the like, or any combination thereof. As another example, the subject may be a man-made composition of organic and/or inorganic matters that are with or without life.

In some embodiments, the scan information may include information relating to a scan and/or the subject. For example, the scan information may include value(s) or value range(s) of at least one scanning parameter (e.g., a voltage of a radiation source, a current of the radiation source, an exposure time of the scan, a table moving speed, a gantry rotation speed, a field of view (FOV), a distance between the radiation source and a detector, a collimation width, a pitch, a rotation time), a scan region of the subject, a scan mode, a shape of a beam limiter (e.g., a shape of a radiation field formed by leaves of a collimator), feature information of the subject, a thickness of a scan layer (e.g., a CT scan layer) of a medical device (e.g., a CT device), a radiation dose of the scan region, a radiation dose of each layer of a plurality of layers of the scan region, a radiation dose of each position of a plurality of positions of the scan region, a radiation dose of a reference region of the scan region, or the like, or any combination thereof.

The scan mode may be defined by a medical device that scans the subject. For example, the scan model may include a CT scan (e.g., a spiral CT scan, a plain CT scan), a digital radiography (DR) scan, an MRI scan, a PET scan, a beam limited scan, or the like, or any combination thereof.

As used herein, a scan region refers to a body region (e.g., an organ, tissue) of the subject to be scanned (e.g., imaged or treated) by a medical device. For example, the scan region may include the head, the neck, the throat, the chest, the abdomen, a hand, a leg, a foot, a spine, a pelvis, a hip, or the like, or any combination thereof, of the subject. In some embodiments, the scan region may include a plurality of layers. Each layer of the plurality of layers may correspond to a scan layer (e.g., a CT scan layer) of the medical device. For example, a thickness of the each layer of the plurality of layers may correspond to the thickness of a scan layer (e.g., a CT scan layer) of the medical device. In some embodiments, a collimator of the medical device may control a width of radiation beams (e.g., X-ray beams) in a scan axis direction (e.g., the Z-axis direction as illustrated in FIG. 1), so as to determine the thickness of the scan layer of the medical device.

In some embodiments, the scan region may include a reference region. As used herein, a reference region refers to a region (e.g., an organ, tissue, a body portion) that is (substantially) not (or not expected to be) exposed to radiation or is exposed to relatively less radiation. In some embodiments, the reference region may include a region (e.g., an organ, tissue, a body portion) that is easily affected by or sensitive to radiation. For example, the reference region may include an eye, a belly button, or the like.

In some embodiments, the radiation dose may indicate the amount of radiation delivered to one or more portions of the subject and/or an absorbed dose that is absorbed by the one or more portions of the subject. For example, the radiation dose may indicate the amount of radiation per unit area delivered to the subject and/or the amount of radiation per unit area absorbed by the subject. As another example, the radiation dose may indicate a total amount of the radiation delivered to the subject and/or a total amount of the radiation absorbed by the subject.

In some embodiments, the radiation dose may be defined by a CT dose index (CTDI) (e.g., a volume CT dose index (CTDIvoi), a weighted CT dose index (CTDIw)), an effective dose, a dose-length product (DLP), or the like. The CT dose index (CTDI) may refer to the radiation energy of radiation corresponding to a single slice along a scan axis direction (e.g., the Z-axis direction as illustrated in FIG. 1) of the medical device (e.g., a CT device). The dose-length product may refer to a product of CT dose index and a scanning range. The effective dose may refer to the radiation energy of radiation received by the subject during a scan.

In some embodiments, the radiation dose (e.g., the radiation dose of the scan region, the radiation dose of each layer of the plurality of layers of the scan region, the radiation dose of each point of the plurality of points of the scan region, the radiation dose of the reference region of the scan region) may be determined based on a value of at least one scanning parameter relating to a scan of the subject. The at least one scanning parameter may include a voltage of a radiation source of the medical device, a current of the radiation source, an exposure time of the scan, a table moving speed, a gantry rotation speed, a field of view (FOV), a distance between the radiation source and a detector (also referred to as a source image distance, or an SID), or the like, or any combination thereof. In some embodiments, the processing device 120 may determine the radiation dose based on a relationship between the radiation dose and the at least one scanning parameter. In some embodiments, the relationship between the radiation dose and the at least one scanning parameter may be determined by performing a plurality of reference scans on a reference subject. The reference subject may be the same as or different from the subject. For example, the processing device 120 may obtain a plurality of sets of reference values of the at least one scanning parameter. Each set of the plurality of sets of reference values may include a reference value of each of the at least one scanning parameter. For each set of the plurality of sets of reference values, a medical device (e.g., the medical device 110) may perform a reference scan on the reference subject according to the set of reference values, and a value of the radiation dose may be measured during the reference scan. For example, the reference subject may be the air, and a radiation dosimeter may be used to measure the value of the radiation dose during the reference scan. The processing device 120 may determine the relationship between the radiation dose and the at least one scanning parameter based on the plurality of sets of reference values of the at least one scanning parameter and the plurality of values of the radiation dose corresponding to the plurality of sets of reference values.

In some embodiments, the processing device 120 may determine the relationship between the radiation dose and the at least one scanning parameter by performing at least one of a mapping operation, a fitting operation, a model training operation, or the like, or any combination thereof, on the plurality of sets of reference values of the at least one scanning parameter and the plurality of values of the radiation dose corresponding to the plurality of sets of reference values. For example, the relationship may be presented in the form of a table recording the plurality of sets of reference values of the at least one scanning parameter and their corresponding values of the radiation dose. As another example, the relationship may be presented in the form of a fitting curve or a fitting function that describes how the value of the radiation dose changes with the reference value of the at least one scanning parameter. As still another example, the relationship may be presented in the form of a dose estimation model. A plurality of training samples may be generated based on the sets of reference values of the at least one scanning parameter and their corresponding values of the radiation dose. The dose estimation model may be configured to determine a value of the radiation dose based on value(s) of the scanning parameter(s).

In some embodiments, the processing device 120 may determine the radiation dose (e.g., a dose distribution) based on feature information of the subject and a value of at least one scanning parameter using a dose estimation algorithm. Exemplary feature information may include a body shape, a height, a width, a thickness, an attenuation coefficient, or the like, or any combination thereof, of the subject. Exemplary dose estimation algorithms may include a Monte Carlo algorithm, a greedy algorithm, a dynamic programming algorithm, a divide-and-conquer algorithm, a backtracking algorithm, a branch bound algorithm, a pencil beam algorithm, a cone convolution algorithm, or the like, or any combination thereof.

In some embodiments, the processing device 120 may determine a dose distribution using a Monte Carlo algorithm. For example, the processing device 120 may determine information of a plurality of radiation particles emitted from a radiation source of the medical device based on one or more scanning parameters related to the radiation source. The information of the plurality of radiation particles may include an energy of each radiation particle, a moving direction of the each radiation particle, a position of the each radiation particle, a number (or count) of the plurality of radiation particles, or the like, or any combination thereof. The processing device 120 may simulate a transport process of each radiation particle of the plurality of radiation particles based on one or more physical processes that may occur during the transport process of the each radiation particle. The one or more physical processes may include a collision between the radiation particle and an atom (or a portion thereof) in a medium (e.g., the air, a tissue of the subject, etc.) that the radiation particle is penetrating, a change in the energy of the radiation particle after the collision, a generation of secondary particles (e.g., electrons) after the collision, a change in the moving direction of the radiation particle, or the like, or any combination thereof. The processing device 120 may determine an energy deposition of the plurality of radiation particles in one or more portions of the subject based on the information of the plurality of radiation particles, the transport process of the each radiation particle of the plurality of radiation particles, and/or the feature information of the subject. The processing device 120 may determine the dose distribution based on the energy deposition of the plurality of radiation particles in one or more portions of the subject. In some embodiments, the processing device 120 may determine the radiation dose of the scan region, the radiation dose of each layer of the plurality of layers of the scan region, the radiation dose of each point of the plurality of points of the scan region, and/or the radiation dose of the reference region based on the dose distribution.

In some embodiments, the scan information (e.g., value(s) or value range(s) of the at least one scanning parameter, the scan region of the subject, and/or the scan mode) may be previously determined and stored in a storage device (e.g., the storage device 130, the storage device 220, the storage 390, or an external source). The processing device 120 may retrieve the scan information from the storage device. Additionally or alternatively, the scan information (e.g., the feature information of the subject, the radiation dose of the scan region, the radiation dose of each layer of a plurality of layers of the scan region, the radiation dose of each position of a plurality of positions of the scan region, and/or the radiation dose of the reference region of the scan region) may be determined by a user (e.g., a doctor) of the medical system 100 or one or more components (e.g., the processing device 120) of the medical system 100. For example, before a scan of the subject, a user (e.g., a doctor) of the medical system 100 may set the scan mode and/or the value(s) of the at least one scanning parameter. The medical device 110 may perform the scan on the scan region of the subject according to the value(s) of the at least one scanning parameter. After the scan of the subject, the processing device 120 may obtain the scan mode, the scan region, and/or the value(s) of the at least one scanning parameter from the medical device 110. The processing device 120 may determine the radiation dose of the scan region based on the value(s) of the at least one scanning parameter.

In 520, the processing device 120 (e.g., the determination module 420) may determine a record mode based on the scan mode.

In some embodiments, the record mode may reflect a distribution of the radiation dose in at least one dimension (e.g., the X-axis direction, the Y-axis direction, the Z-axis direction as illustrated in FIG. 1) of the scan region of the subject. The record mode may refer to a presentation form of the radiation dose or the distribution of the radiation dose. In some embodiments, the record mode may include a radiation dose curve, a radiation dose model, a radiation dose table, a radiation dose chart (e.g., a bar chart, a pie chart, a scatter chart, a radar chart), or the like, or any combination thereof.

In some embodiments, the processing device 120 may determine the record mode based on the scan mode. For example, the processing device 120 may determine the record mode based on the scan mode and a relationship between the record mode and the scan mode. The relationship between the record mode and the scan mode may be manually set by a user (e.g., a doctor) of the medical system 100, or by one or more components (e.g., the processing device 120) of the medical system 100 according to different situations. The relationship between the record mode and the scan mode may be stored in a storage device (e.g., the storage device 130) of the medical system 100, and the processing device 120 may retrieve the relationship between the record mode and the scan mode from the storage device. As another example, the processing device 120 may add the scan mode and the record mode corresponding to the scan mode in software codes of radiation dose management.

In 530, the processing device 120 (e.g., the recording module 430) may record the scan region and the radiation dose based on the record mode.

In some embodiments, the scan mode may include a spiral CT scan. In the spiral CT scan, a spiral CT device may scan the subject in a spiral path. For example, during the spiral CT scan, the subject supported on a scanning table may move along a scan axis direction (e.g., the Z-axis direction as illustrated in FIG. 1) of the spiral CT device continuously, and a radiation source (e.g., a tube) and a detector connected to a gantry of the spiral CT device may rotate around a rotation axis (e.g., the Z-axis direction as illustrated in FIG. 1) of the spiral CT device to scan the subject. The processing device 120 may determine the record mode as a radiation dose curve based on the spiral CT scan. The radiation dose curve may be configured to reflect radiation doses corresponding to a plurality of layers of the scan region.

In some embodiments, the processing device 120 may determine a radiation dose of each layer of the plurality of layers of the scan region. For example, the processing device 120 may determine the radiation dose of the each layer of the plurality of layers of the scan region based on a value of at least one scanning parameter as described in connection with operation 510. In some embodiments, the plurality of layers may be contiguous. The processing device 120 may determine the radiation dose curve based on the radiation doses of the plurality of layers of the scan region. The processing device 120 may record the radiation doses corresponding to the plurality of layers of the scan region using the radiation dose curve. For example, the processing device 120 may determine a plurality of points based on radiation doses of the plurality of layers of the scan region and positions of the plurality of layers of the scan region. The plurality of points may be used to form the dose curve. Each point may correspond to a radiation dose of the each layer of the plurality of layers of the scan region. The processing device 120 may determine the radiation dose curve by connecting the plurality of points.

In some embodiments, in the spiral CT scan, the spiral CT device may scan a first region adjacent to the scan region and a second region adjacent to the scan region. For example, before the spiral CT device scans the scan region, the gantry including the radiation source (e.g., the tube) and the detector may rotate 0.25-0.5 circle in the first region and scan the first region. After the spiral CT device scans the scan region, the gantry including the radiation source (e.g., the tube) and the detector may rotate 0.25-0.5 circle in the second region and scan the second region. Accordingly, the radiation dose curve may include three sections corresponding to scan processes of the first region, the scan region, and the second region.

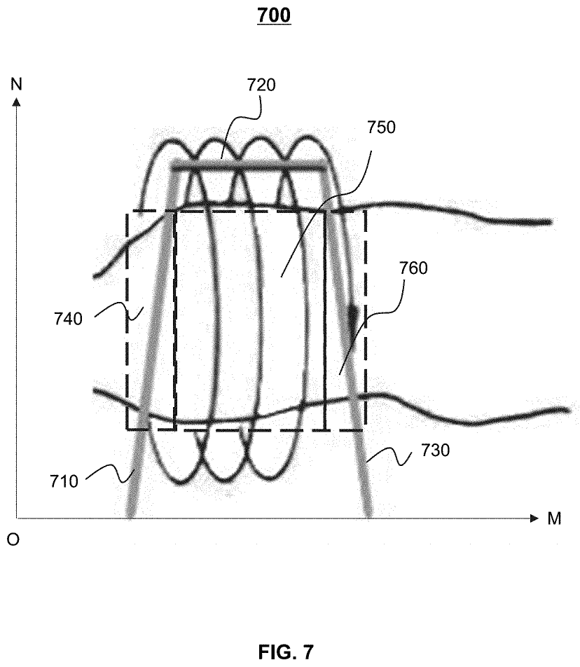

FIG. 7 is a schematic diagram illustrating an exemplary radiation dose curve according to some embodiments of the present disclosure. As illustrated in FIG. 7, an M-axis of a radiation dose curve 700 may represent a position (e.g., the Z-axis coordinate of the position) of the scan region along a scan axis direction (e.g., the Z-axis direction as illustrated in FIG. 1) of the spiral CT device. An N-axis of the radiation dose curve 700 may represent a radiation dose. The radiation dose curve 700 may include a first section 710, a second section 720, and a third section 730. The first section 710 may correspond to a scan process of a first region 740. The second section 720 may correspond to a scan process of a scan region 750. The third section 730 may correspond to a scan process of a second region 760. For example, the first section 710 may be a straight line representing the radiation dose gradually changes from zero to a target radiation dose (e.g., 80 mGy/cm). That is, radiation doses of one or more layers of the first region 740 may gradually change from zero to the target radiation dose. The second section 720 may be a straight line representing the radiation dose maintains the target radiation dose. That is, radiation doses of one or more layers of the scan region 750 may maintain the target radiation dose. The third section 730 may be a straight line representing the radiation dose gradually changes from the target radiation dose to zero. That is, radiation doses of one or more layers of the second region 760 may gradually change from the target radiation dose to zero. It should be noted that the above description of the radiation dose curve 700 is merely provided for the purposes of illustration, and not intended to limit the scope of the present disclosure. In some embodiments, the radiation dose curve may include one or more straight lines, one or more curved lines, or the like. In some embodiments, the radiation dose curve 700 may correspond to a time point in the scan of the subject. For example, the radiation dose curve 700 may reflect radiation doses of the plurality of layers of the scan region corresponding to a specific time point in the scan of the subject. In some embodiments, a plurality of radiation dose curves corresponding to a plurality of time points in the scan of the subject may be generated. The radiation dose curves corresponding to different time points in the scan of the subject may be the same or different.

In some embodiments, a collimator of the spiral CT device may be used to control a radiation region (i.e., a radiation field) on the subject, to prevent radiation beams emitted from the radiation source (e.g., the tube) from irradiating outside the scan region of the subject. In a traditional way, when recording the radiation dose of the scan region of the subject, radiation doses of regions (e.g., the first region, the second region) adjacent to the scan region may usually be regarded as the radiation dose of the scan region. Accordingly, the radiation dose of the scan region determined by the traditional way may be different from (e.g., less than or greater than) an actual radiation dose of the scan region. In addition, in the traditional way, when recording the radiation dose of the scan region of the subject, only a value of an average radiation dose and/or a value of a dose-length product of the scan region may be determined. According to some embodiments of the present disclosure, the radiation dose of the scan region may be represented as a radiation dose curve. The radiation dose curve may show radiation doses of a plurality of positions (e.g., a plurality of layers) of the regions (e.g., the first region, the second region) adjacent to the scan region and the scan region. Therefore, the radiation dose of the scan region of the subject may be presented intuitively and accurately.

In some embodiments, the scan mode may include a CT scan (e.g., a plain CT scan, a spiral CT scan). In some embodiments, in the CT scan, the subject (e.g., a patient) may be equivalent to a phantom including one or more elliptical cylinders based on feature information (e.g., the height, the thickness, the width) of the subject and/or a topogram image of the subject. In some embodiments, the topogram image may be associated with a localizer scan. The localizer scan may be performed by a medical device (e.g., a CT device) when a radiation source is in a stationary position and a scanning table moves along the scan axis direction (e.g., the Z-axis direction as illustrated in FIG. 1). As used herein, a width of a subject refers to a length of the subject (e.g., a length at the center of the subject, a maximum length of the subject) along a direction perpendicular to a sagittal plane of the subject. A height of a subject refers to a length of the subject (e.g., a length at the center of the subject, a maximum length of the subject) along a direction perpendicular to a transverse plane of the subject. A thickness of a subject refers to a length of the subject (e.g., a length at the center of the subject, a maximum length of the subject) along a direction perpendicular to a coronal plane of the subject.

In some embodiments, since the width of the subject is usually larger than the thickness of the subject, in the CT scan, when the subject is lying on the scanning table, the CT device may scan the subject with a relatively large radiation dose in a first axis direction (to facilitate radiation penetration through the width of the subject), and scan the subject with a relatively small radiation dose in a second axis direction (to facilitate radiation penetration through the thickness of the subject). The first axis may be perpendicular to a sagittal plane of the subject. The second axis may be perpendicular to a coronal plane of the subject. For example, the radiation source positioned on a side of the subject may scan the subject with a relatively large radiation dose, while the radiation source positioned above the subject may scan the subject with a relatively small radiation dose. Accordingly, for a specific layer of a plurality of layers of the scan region of the subject, the radiation doses corresponding to a plurality of positions of the specific layer may correspond to the distances between a center point of an ellipse and a plurality of points on a perimeter of the ellipse. For example, as illustrated in FIG. 9, the radiation dose across the width of the subject may be larger than the radiation dose across the thickness of the subject, and accordingly, the distance between the point O and the point a representing the radiation dose across the width of the subject may be larger than the distance between the point O and the point b representing the radiation dose across the thickness of the subject. The radiation dose of each layer of the plurality of layers of the scan region of the subject may correspond to an area of a corresponding ellipse.

The processing device 120 determine the record mode as a first model including a plurality of elliptical cylinders based on the CT scan. The first model may be configured to reflect radiation doses corresponding to a plurality of layers of the scan region. The first model may also be configured to reflect radiation doses corresponding to a plurality of positions on each layer of the plurality of layers of the scan region.

In some embodiments, the processing device 120 may determine a radiation dose of the each layer of the plurality of layers of the scan region. The processing device 120 may generate the first model based on the thickness of the each layer of the plurality of layers of the scan region. The height of each elliptical cylinder of the plurality of elliptical cylinders may correspond to a thickness of a corresponding layer of the plurality of layers of the scan region. For example, a relatively large height of an elliptical cylinder may correspond to a relatively large thickness of a corresponding layer. A relatively small height of an elliptical cylinder may correspond to a relatively small thickness of a corresponding layer. The height of each two elliptical cylinders of the plurality of elliptical cylinders may be the same or different. A cross-sectional area of the each elliptical cylinder may correspond to a radiation dose of the corresponding layer. The processing device 120 may record the radiation doses corresponding to the plurality of layers of the scan region using the first model. In some embodiments, a cross section of each elliptical cylinder may have an ellipse shape. The ellipse may have a major axis and a minor axis. The minor axis of each ellipse may be the same. A length of the major axis of the each ellipse may correspond to a radiation dose of a corresponding layer. For example, a relatively large length of a major axis of an ellipse may correspond to a relatively large radiation dose of a corresponding layer. In some embodiments, the major axis of the each ellipse may be the same. A length of the minor axis of the each ellipse may correspond to a radiation dose of the corresponding layer. For example, a relatively large length of a minor axis of an ellipse may correspond to a relatively large radiation dose of a corresponding layer.

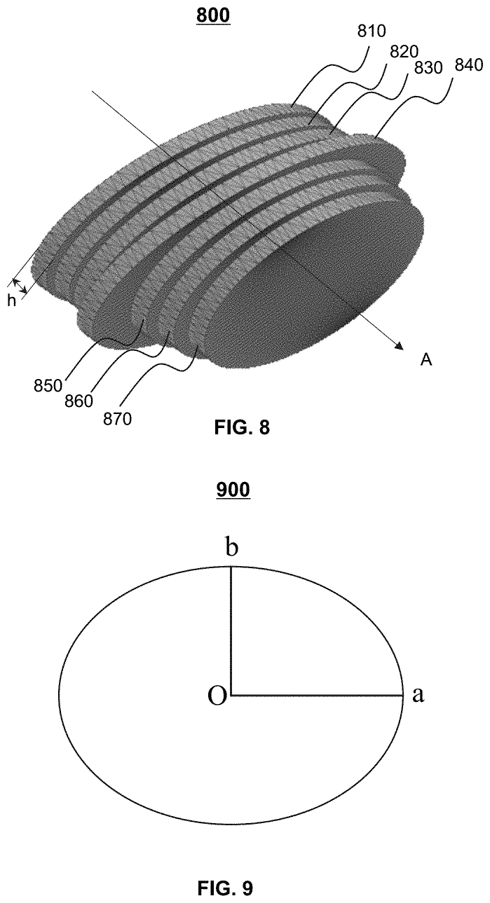

FIG. 8 is a schematic diagram illustrating an exemplary first model according to some embodiments of the present disclosure. As illustrated in FIG. 8, a first model 800 may include a plurality of elliptical cylinders (e.g., an elliptical cylinder 810, an elliptical cylinder 820, an elliptical cylinder 830, an elliptical cylinder 840, an elliptical cylinder 850, an elliptical cylinder 860, an elliptical cylinder 870). The height of each elliptical cylinder (e.g., the height h of the elliptical cylinder 810) of the plurality of elliptical cylinders may correspond to a thickness of a corresponding layer of the plurality of layers of the scan region. In some embodiments, a longitudinal axis A of the each elliptical cylinder of the plurality of elliptical cylinders may be located on a same straight line. For example, the longitudinal axis A of the each elliptical cylinder of the plurality of elliptical cylinders may be parallel to a scan axis direction (e.g., the Z-axis direction as illustrated in FIG. 1) of a CT device. In some embodiments, the longitudinal axis A of the each elliptical cylinder of the plurality of elliptical cylinders may be perpendicular to a cross section of the subject.

FIG. 9 is a schematic diagram illustrating an exemplary elliptical cylinder according to some embodiments of the present disclosure. As illustrated in FIG. 9, a cross section of an elliptical cylinder 900 may have an ellipse shape. The point O may be a projection point of a longitudinal axis of the elliptical cylinder 900 on the cross section of the elliptical cylinder 900. A distance between the point O and a point on the elliptical cylinder 900 may correspond to a radiation dose of a corresponding position of the scan region of the subject. For example, a greater distance between the point O and a point (e.g., the point a) on the elliptical cylinder 900 may correspond to a larger value of a radiation dose of a corresponding position of the scan region of the subject. A smaller distance between the point O and a point (e.g., the point b) on the elliptical cylinder 900 may correspond to a smaller value of a radiation dose of a corresponding position of the scan region of the subject. For example, a distance between the point O of the elliptical cylinder 900 and a point on a perimeter of the elliptical cylinder 900 may correspond to a radiation dose of a corresponding position on an edge of a corresponding layer of the scan region of the subject.

In some embodiments, the scan mode may include a CT scan (e.g., a plain CT scan, a spiral CT scan). The scan region may include a reference region. The processing device 120 may determine the record mode as a second model based on the CT scan and the reference region. The second model may include a cylinder with a first curved surface. The first curved surface may correspond to the reference region.

In some embodiments, the processing device 120 may generate the cylinder based on the scan region and the radiation dose of the scan region. In some embodiments, a longitudinal axis of the cylinder may be perpendicular to a cross section of the subject. A heigh of the cylinder may correspond to a length of the scan region along a scan axis direction (e.g., the Z-axis direction as illustrated in FIG. 1) of the CT device. A cross-sectional area of the cylinder may correspond to the radiation dose of the scan region. For example, a relatively large cross-sectional area of the cylinder may correspond to a relatively large radiation dose of the scan region. The processing device 120 may generate the second model by forming, based on the radiation dose of the reference region, the first curved surface on the cylinder. In some embodiments, the reference region may include a region (e.g., an organ, tissue, a body portion) that is easily affected by or sensitive to radiation. The radiation dose of the reference region may need to be controlled (or minimized or avoided) to avoid damaging the reference region. For example, a position of the collimator (e.g., positions of leaves of the collimator) of the medical device (e.g., the CT device) may be adjusted to ensure that radiation beams (e.g., X-ray beams) emitted from the radiation source (e.g., the tube) cannot directly irradiate the reference region. As another example, the medical device (e.g., the CT device) may scan the reference region with a relatively small radiation dose. Accordingly, the reference region may only receive radiation attenuated by other body regions of the subject, which may avoid damaging the reference region while ensuring the image quality of the scan region of the subject.

In some embodiments, the first curved surface may have a cone shape, a pyramid shape, or the like. A distance between a position of the first curved surface and the longitudinal axis of the cylinder may correspond to a radiation dose of a corresponding position of the reference region. In some embodiments, the distance between a position of the first curved surface and the longitudinal axis of the cylinder may correspond to a radiation dose of a corresponding position on an edge of the reference region. For example, a relatively large distance between a position of the first curved surface and the longitudinal axis of the cylinder may correspond to a relatively large radiation dose of a corresponding position of the reference region. The processing device 120 may record radiation doses corresponding to a plurality of positions of the reference region using the second model. In some embodiments, the reference region may include a plurality of layers. The processing device 120 may obtain a radiation dose of each layer of the plurality of layers of the reference region. The processing device 120 may generate the first curved surface with a cone shape including a plurality layers based on the thickness of the each layer of the plurality of layers of the reference region, and the radiation dose of the each layer of the plurality of layers of the reference region. A height of each layer of the plurality of layers of the first curved surface may correspond to a thickness of a corresponding layer of the plurality of layers of the reference region. A cross-sectional area of the each layer of the plurality of layers of the first curved surface may correspond to a radiation dose of the corresponding layer of the plurality of layers of the reference region.

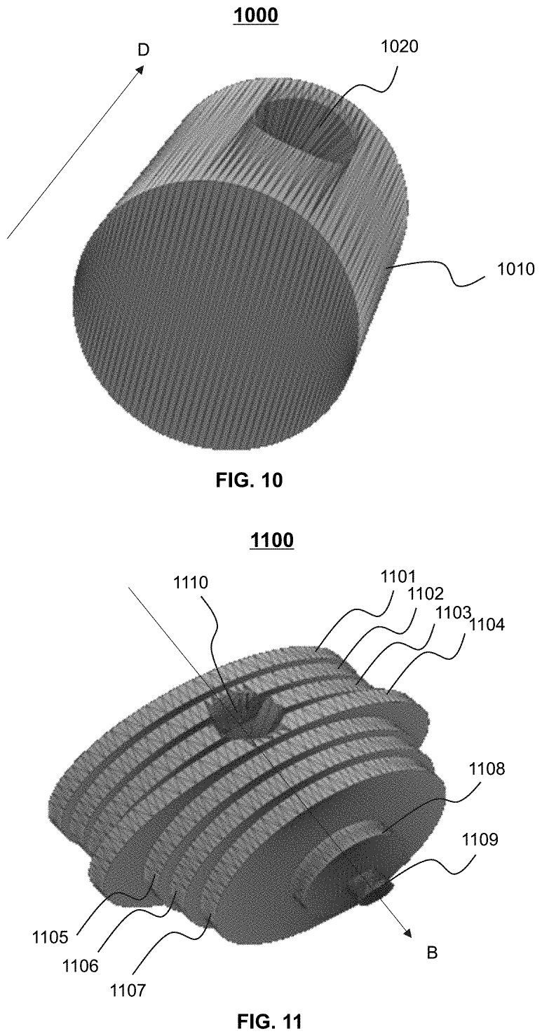

FIG. 10 is a schematic diagram illustrating an exemplary second model according to some embodiments of the present disclosure. As illustrated in FIG. 10, a second model 1000 may include a cylinder 1010 with a first curved surface 1020. The first curved surface 1020 may correspond to a reference region of the subject. In some embodiments, a longitudinal axis D of the cylinder 1010 may be perpendicular to a cross section of a subject, and parallel to the scan axis direction (e.g., the Z-axis direction as illustrated in FIG. 1) of the medical device.

In some embodiments, the scan mode may include the CT scan. The scan region may include the reference region. The processing device 120 may determine the record mode as a third model based on the CT scan and the reference region. The third model may include a plurality of elliptical cylinders with a second curved surface. The second curved surface may correspond to the reference region. The third model may be configured to reflect radiation doses corresponding to a plurality of layers of the scan region.

In some embodiments, the processing device 120 may determine the radiation dose of each layer of the plurality of layers of the scan region. The processing device 120 may generate the plurality of elliptical cylinders based on the thickness of the each layer of the plurality of layers of the scan region, and the radiation dose of the each layer of the plurality of layers of the scan region. A longitudinal axis of the each elliptical cylinder of the plurality of elliptical cylinders may be located on a same straight line. The longitudinal axis of the each elliptical cylinder of the plurality of elliptical cylinders may be perpendicular to a cross section of the subject. A height of the each elliptical cylinder of the plurality of elliptical cylinders may correspond to a thickness of a corresponding layer of the plurality of layers of the scan region. A cross-sectional area of the each elliptical cylinder may correspond to a radiation dose of the corresponding layer. The generation of the plurality of elliptical cylinders of the third model may be similar to the generation of the plurality of elliptical cylinders of the first model as described elsewhere in the present disclosure. The processing device 120 may generate the third model by forming, based on the radiation dose of the reference region, the second curved surface on the plurality of elliptical cylinders. The forming of the second curved surface in the third model may be similar to the forming of the first curved surface in the second model as described elsewhere in the present disclosure. The processing device 120 may record radiation doses corresponding to the plurality of layers of the scan region and a plurality of positions of the reference region using the third model.

FIG. 11 is a schematic diagram illustrating an exemplary third model according to some embodiments of the present disclosure. As illustrated in FIG. 11, a third model 1100 may include a plurality of elliptical cylinders (e.g., an elliptical cylinder 1101, an elliptical cylinder 1102, an elliptical cylinder 1103, an elliptical cylinder 1104, an elliptical cylinder 1105, an elliptical cylinder 1106, an elliptical cylinder 1107, an elliptical cylinder 1108, an elliptical cylinder 1109) with a second curved surface 1110. The height of each elliptical cylinder of the plurality of elliptical cylinders may correspond to a thickness of a corresponding layer of the plurality of layers of the scan region of the subject. In some embodiments, a longitudinal axis B of the each elliptical cylinder of the plurality of elliptical cylinders may be located on a same straight line. In some embodiments, the longitudinal axis B of the each elliptical cylinder of the plurality of elliptical cylinders may be perpendicular to a cross section of the subject, and parallel to the scan axis direction (e.g., the Z-axis direction as illustrated in FIG. 1) of the medical device. The second curved surface 1110 may correspond to a reference region of the subject. In some embodiments, a distance between a position of the second curved surface 1110 and the longitudinal axis B of the plurality of elliptical cylinders may correspond to a radiation dose of a corresponding position of the reference region.

It should be noted that the above description is merely provided for the purposes of illustration, and not intended to limit the scope of the present disclosure. In some embodiments, the first model, the second model, and/or the third model may have other shapes. For example, the first model, the second model, and/or the third model may be a cuboid.

In some embodiments, the scan mode may include a beam limited scan mode. In the beam limited scan mode, a beam limiter (e.g., a collimator) may be used to control a radiation region (i.e., a radiation field) on the subject. For example, the beam limiter may control a shape of radiation beams emitted from the radiation source of the medical device. In some embodiments, the beam limited scan mode may be performed by an X-ray device (e.g., a C-arm device, a mamo X-ray device). The processing device 120 may determine the record mode as a fourth model based on the beam limited scan mode. In some embodiments, the fourth model may include a frustum (e.g., a frustum of a cone, a frustum of a pyramid).

In some embodiments, the processing device 120 may generate the fourth model based on the shape of the beam limiter and the radiation dose of the scan region. An axis of the frustum may be perpendicular to a coronal plane of the subject. The axis of the frustum may be parallel to a beam axis of the radiation beams emitted from the radiation source of the medical device. A shape of a surface of the frustum may correspond to the shape of the beam limiter. For example, the shape of the surface of the frustum may be the same as the shape of a radiation field formed by leaves of a multi-leaf collimator. In some embodiments, for the beam limited scan mode performed by the C-arm device or the mamo X-ray device that scans around the subject, the shape of the surface of the frustum may correspond to the shape of the beam limiter at a specific position and/or scanning angle. A height of the frustum may correspond to the radiation dose of the scan region. For example, a relatively large radiation dose of the scan region may correspond to a relatively large height of the frustum. In some embodiments, a distance between a position of the frustum and the surface of the frustum may correspond to a radiation dose of a corresponding position on the reference region. For example, a relatively large distance between a position of the frustum and the surface of the frustum may correspond to a relatively large radiation dose of a corresponding position of the reference region. In some embodiments, the processing device 120 may record the radiation dose of the scan region using the fourth model.

In some embodiments, the processing device 120 may generate a subject model representing at least a portion of the subject based on feature information of the subject and at least one image (e.g., a CT image) of the subject, as described elsewhere in the present disclosure (e.g., operation 620 in FIG. 6, and descriptions thereof). The processing device 120 may determine CT values (e.g., Hounsfield values) of a plurality of voxels of the scan region of the subject in the at least one image (e.g., the CT image) of the subject. The processing device 120 may determine the radiation dose of the scan region based on the CT values (e.g., Hounsfield values) of the plurality of voxels of the scan region of the subject. The processing device 120 may generate the frustum based on a beam axis of the radiation beams emitted from the radiation source of the medical device, the radiation dose of the scan region, and/or the shape of the beam limiter.

In some embodiments, the processing device 120 may obtain a dose-volume histogram (DVH) of the subject. The processing device 120 may determine a dose distribution based on the feature information of the subject, at least one image (e.g., a CT image) of the subject, and the dose-volume histogram (DVH) of the subject using a Monte Carlo algorithm. The processing device 120 may generate the frustum based on the dose distribution, the scan region, and/or the feature information of the subject.

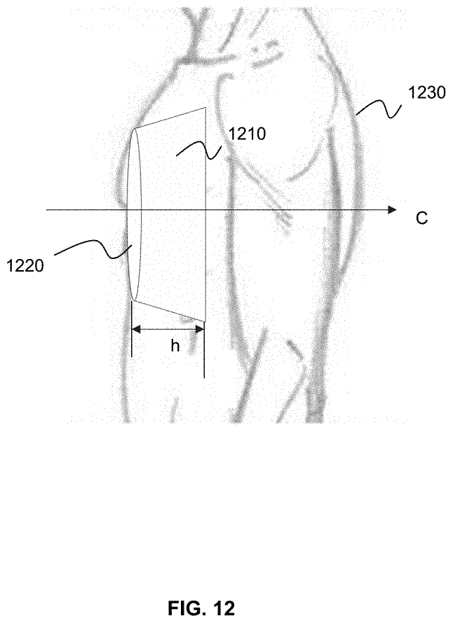

FIG. 12 is a schematic diagram illustrating an exemplary fourth model according to some embodiments of the present disclosure. As illustrated in FIG. 12, a fourth model 1210 may include a frustum of a cone. An axis C of the frustum 1210 may be perpendicular to a coronal plane of a subject 1230. A shape of a surface 1220 of the frustum 1210 may correspond to a shape of a beam limiter of a medical device. A height h of the frustum 1210 may correspond to a radiation dose of a scan region of the subject 1230.

It should be noted that the above description is merely provided for the purposes of illustration, and not intended to limit the scope of the present disclosure. In some embodiments, the processing device 120 may determine the scan model as any combination of the radiation dose curve, the first model, the second model, the third model, and the fourth model. For example, the processing device 120 may determine a radiation dose curve and a first model including a plurality of elliptical cylinders based on a spiral CT scan.

In some embodiments, the processing device 120 may generate a model based on the record mode. The processing device 120 may visualize the scan region and the radiation dose using the model. For example, the processing device 120 may generate a subject model representing at least a portion of the subject. The processing device 120 may generate the model by mapping the scan region, and the radiation dose of the scan region on the subject model according to the record mode. The processing device 120 may cause a terminal device to display the model visualizing the scan region and the radiation dose. In some embodiments, a user (e.g., a doctor) of the medical system 100 may adjust (e.g., modify, zoom in, zoom out) the model displayed on an interface of the terminal device. For example, the user may select an elliptical cylinder of the plurality of elliptical cylinders of the first model to obtain radiation doses of a plurality of positions of the elliptical cylinder. More descriptions of the visualizing the scan region and the radiation dose may be found elsewhere in the present disclosure (e.g., FIG. 6 and descriptions thereof).

According to some embodiments of the present disclosure, a distribution of the radiation dose in at least one dimension of the scan region of the subject during a scan may be visualized using one or more models (e.g., the first model, the second model, the third model, the fourth model). A user (e.g., a doctor) of the medical system 100 may view the distribution of the radiation dose of the subject intuitively, and/or manage the distribution of the radiation dose of the subject effectively. For example, the position of the reference region, the radiation dose of the reference region, and/or radiation doses corresponding to the plurality of layers of the scan region may be presented accurately and intuitively. Therefore, the accuracy and/or efficiency of radiation dose management may be improved.

In addition, the radiation doses corresponding to a plurality of portions of the scan region may be presented accurately and intuitively using a visualization model (e.g., the first model, the second model, the third model, the fourth model). For example, a scan may be performed on a first scan region and a second scan region, or two scans may be performed respectively on the first scan region and the second scan region. The first scan region and the second scan region may have an overlap region. Traditionally, a radiation dose of the first scan region and a radiation dose of the second scan region may be determined based on the scan information of the first scan region and the second region. However, a radiation dose of the overlap region may not be determined. According to some embodiments of the present disclosure, the radiation doses corresponding to a plurality of portions (e.g., a plurality of layers) of the scan region may be recorded and/or visualized using the visualization model, and the radiation dose of the overlap region may be determined or presented based on the visualization model. A scan protocol (e.g., a value of at least one scanning parameter, a scan mode) associated with a next scan of the subject may be determined and/or adjusted based on the radiation dose of the overlap region, which may prevent the overlap region from receiving excessive radiation.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, process 500 may be used in a hospital information system (HIS), a laboratory information management system (LIS), a picture archiving and communication systems (PACS), a radiology information system (RIS), or the like, or any combination thereof.

In some embodiments, the record mode may reflect a distribution of the radiation dose in two dimensions (2D) or three dimensions (3D) of the scan region of the subject. In some embodiments, the radiation dose of the scan region may be represented by a color. For example, the record mode may include a 3D model. The color of each point of the 3D model may correspond to a radiation dose of a corresponding position (e.g., a voxel) of the scan region of the subject.

In some embodiments, process 500 may be performed before and/or after a scan of the subject. For example, before a scan of the subject, the processing device 120 may determine an estimated distribution of a radiation dose in at least one dimension of a scan region of the subject based on scan information of the subject. The processing device 120 may provide the estimated distribution of the radiation dose in a visualization model of at least a portion of the subject. The processing device 120 (or a user) may determine whether an estimated radiation dose of at least one portion of the scan region exceeds a corresponding dose threshold based on the estimated distribution of the radiation dose. In response to determining that an estimated radiation dose of a portion of the scan region exceeds a corresponding dose threshold, the processing device 120 may generate a reminder. The reminder may be in the form of text, voice, a picture, a video, a haptic alert, or the like, or any combination thereof. In some embodiments, the reminder may indicate that the estimated radiation dose of which portion(s) of the scan region exceeds the corresponding dose threshold. The processing device 120 (or the user) may determine and/or adjust a scan protocol of the subject based on the reminder. As another example, after a scan of the subject, the processing device 120 may determine an actual distribution of a radiation dose in at least one dimension of a scan region of the subject based on scan information of the subject. The processing device 120 may provide the actual distribution of the radiation dose in a visualization model of at least a portion of the subject. The processing device 120 may determine and/or adjust a scan protocol of a next scan of the subject based on the visualization model, as described elsewhere in the present disclosure (e.g., FIG. 6 and descriptions thereof). In some embodiments, the scan information may include historical scan information of at least one historical scan of the subject. The processing device 120 may display a current distribution of an accumulative radiation dose corresponding to the at least one historical scan of the subject in the model. The current distribution of the accumulative radiation dose corresponding to the at least one historical scan of the subject may correspond to a current time point. For example, the processing device 120 may determine the current distribution of the accumulative radiation dose corresponding to the at least one historical scan based on the historical scan information of the at least one historical scan of the subject and time information of the at least one historical scan as described elsewhere in the present disclosure (e.g., FIG. 6 and descriptions thereof). In some embodiments, the scan information may include the historical scan information of at least one historical scan of the subject and current scan information of a current scan of the subject. The processing device 120 may display an estimated distribution of an accumulative radiation dose corresponding to the at least one historical scan of the subject and the current scan of the subject in the model.

FIG. 6 is a flowchart illustrating an exemplary process for displaying a subject model, one or more historical scan regions, and one or more historical radiation doses according to one or more historical record modes according to some embodiments of the present disclosure. In some embodiments, process 600 may be executed by the medical system 100. For example, the process 600 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting.

In 610, the processing device 120 (e.g., the obtaining module 410) may obtain one or more historical scan regions of a subject, one or more historical radiation doses of the subject, and one or more historical record modes.

In some embodiments, the processing device 120 may obtain historical scan information of one or more historical scans of the subject. The historical scan information of the one or more historical scans of the subject may include the one or more historical scan regions of the subject, the one or more historical radiation doses of the subject, and the one or more historical record modes. In some embodiments, a historical record mode of a historical scan may be determined based on a historical scan mode of the historical scan according to process 500.

In some embodiments, the historical scan information of the one or more historical scans of the subject may be stored in a storage device (e.g., the storage device 130) of the medical system 100. The processing device 120 may obtain the historical scan information from the storage device (e.g., the storage device 130).

In 620, the processing device 120 (e.g., the determination module 420) may generate a subject model representing the subject.

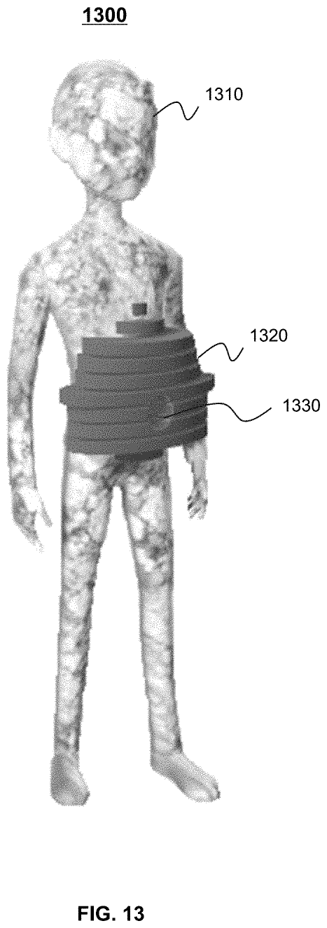

As used herein, a subject model (e.g., a subject model 1310 as illustrated in FIG. 13) refers to a model representing feature information of a subject. In some embodiments, the subject model may include a 2D skeleton model, a 3D skeleton model, a 3D mesh model, or the like, of the subject.

In some embodiments, the processing device 120 may generate the subject model of the subject based on the feature information of the subject. The feature information of the subject may include the gender of the subject, the age of the subject, a shape (e.g., a width, a thickness, a height, a weight) of the subject or a portion thereof, a body mass index (BMI) of the subject, anatomical information associated with organs (or tissue) of the subject, or the like, or any combination thereof. The anatomical information associated with organs (or tissue) of the patient may include the position information of the organs (or tissue) inside the patient, the size information of the organs (or tissue), the shape information of the organs (or tissue), or the like, or any combination thereof.

In some embodiments, the feature information of the subject may be previously determined and stored in a storage device (e.g., the storage device 130, the storage device 220, the storage 390, an external source). The processing device 120 may retrieve the feature information of the subject from the storage device. Additionally or alternatively, the feature information of the subject may be determined based on image data of the subject. For example, an image capturing device (e.g., a camera, a medical device) may capture the image data of the subject, and the processing device 120 may determine the feature information (or a portion thereof) of the subject based on the image data according to an image analysis algorithm (e.g., an image segmentation algorithm, a feature point extraction algorithm). As another example, a radar sensor may capture point cloud data of the subject, and the processing device 120 may determine the feature information (or a portion thereof) of the subject based on the point cloud data of the subject.

In some embodiments, the processing device 120 may obtain a plurality of CT images corresponding to a plurality of layers of the subject. For each CT image of the plurality of CT images, the processing device 120 may perform a preprocessing operation (e.g., an image format conversion operation, a filtering operation) on the CT image to obtain a processed CT image. The processing device 120 may divide the processed CT image into a plurality of regions. Each region may include a same type of voxels. For example, each region may include a plurality of voxels belonging to a same organ or tissue of the subject. The processing device 120 may perform a registration operation and/or an inter-layer interpolation operation on the plurality of regions of a plurality of processed CT images, to obtain the feature information (e.g., the anatomical information) of the subject. In some embodiments, the processing device 120 may obtain a plurality of MRI images of the subject. The feature information (e.g., the anatomical information) of skeletal regions of the subject may be obtained based on the plurality of CT images, and the feature information (e.g., the anatomical information) of soft tissue regions of the subject may be obtained based on the plurality of MRI images, which may improve the accuracy of the feature information of the subject.

In some embodiment, the processing device 120 may generate the subject model (e.g., a 3D mesh model) of the subject based on the feature information of the subject and/or the image data of the subject according to the one or more mesh generation techniques, such as a Triangular/Tetrahedral (Tri/Tet) technique (e.g., an Octree algorithm, an advancing front algorithm, a Delaunay algorithm, etc.), a Quadrilateral/Hexahedra (Quad/Hex) technique (e.g., a Trans-finite Interpolation (TFI) algorithm, an Elliptic algorithm, etc.), a hybrid technique, a parametric model based technique, a surface meshing technique, or the like, or any combination thereof.

In some embodiments, a model library having a plurality of candidate subject models of a plurality of candidate subjects may be previously generated and stored in a storage device (e.g., the storage device 130, the storage device 220, and/or the storage 390, an external source). In some embodiments, the plurality of candidate subjects may include the subject. Registration information of the plurality of candidate subjects may be stored in the storage device. The registration information may include identify information (e.g., an identification (ID) number, a name, the gender, the age, a date of birth, an occupation), contact information (e.g., a mobile phone number), medical information (e.g., a medical record number, a registration card number, a health condition, a medical history), the feature information (e.g., a width, a thickness, a height, a weight) of a candidate subject or a portion thereof, or the like, or any combination thereof.

In some embodiments, the processing device 120 may obtain a candidate subject model of the subject from the model library based on registration information of the subject. The processing device 120 may designate the candidate subject model of the subject as the subject model of the subject. In some embodiments, the processing device 120 may select a candidate subject with the highest degree of similarity to the subject among the plurality of candidate subjects. The processing device 120 may designate the candidate subject model of the selected candidate subject as the subject model of the subject. In some embodiments, the processing device 120 may modify at least part of the candidate subject model of the selected candidate subject based on the feature information of the selected candidate subject and the feature information of the subject, for example, a body shape difference between the candidate subject and the subject. The processing device 120 may further designate the modified candidate subject model as the subject model of the subject.

In some embodiments, the model library may be stored in a hospital information system (HIS), a laboratory information management system (LIS), a picture archiving and communication systems (PACS), a radiology information system (RIS), or the like, or any combination thereof.

In 630, the processing device 120 (e.g., the control module 440) may cause a terminal device (e.g., the terminal device 140) to display the subject model, the one or more historical scan regions and the one or more historical radiation doses according to the one or more historical record modes.

In some embodiments, the processing device 120 may generate target image data by mapping the one or more historical scan regions and the one or more historical radiation doses on the subject model according to the one or more historical record modes. The target image data may include a 2D image, a 3D image, a 4D image, or the like. In some embodiments, in the target image data, one or more representations of the one or more historical record modes (e.g., a radiation dose curve, a first model including a plurality of elliptical cylinders, a second model including a cylinder with a first curved surface, a third model including a plurality of elliptical cylinders with a second curved surface, a fourth model including a frustum, as described in FIG. 5) may be superimposed on a representation of the subject model. FIG. 13 is a schematic diagram illustrating exemplary target image data according to some embodiments of the present disclosure. As illustrated in FIG. 13, in target image data 1300, a representation 1320 of a model including a plurality of elliptical cylinders with a curved surface 1330 may be superimposed on a representation 1310 of a subject model.

In some embodiments, for each of the one or more historical scan regions, the processing device 120 may map the historical scan region on the subject model. The processing device 120 may map the historical radiation dose of the historical scan region on the subject model according to a corresponding historical record mode. For example, the processing device 120 may map the historical record mode recording the historical radiation dose of the historical scan region on the subject model. In some embodiments, the processing device 120 may identify the historical scan region on the subject model. The processing device 120 may map the historical record mode recording the historical radiation dose of the historical scan region on the subject model based on the identified historical scan region and a mapping relationship between a first coordinate system associated with a medical device (e.g., the medical coordinate system 160 as illustrated in FIG. 1) and a second coordinate system associated with the subject model (also referred to as a model coordinate system). The model coordinate system may refer to a coordinate system that describes a position of a point on a subject model. In some embodiments, different model coordinate systems may be constructed for different models. For example, for the model coordinate system of the subject model in some embodiments of the present disclosure, an origin may be a center point of the subject model. The X-axis may be from a left side to a right side of the subject model viewed from a direction facing the front of the subject model. The Y-axis may be from a front side to a rear side of the subject model. The Z axis direction may be from a lower side to an upper side of the subject model. In some embodiments, the mapping relationship between the first coordinate system associated with the medical device and the second coordinate system associated with the subject model may be determined based on position information of one or more components (e.g., a scanning table, a radiation source, a detector) of the medical device, and the feature information of the subject. The mapping relationship between the first coordinate system associated with the medical device and the second coordinate system associated with the subject model may be previously determined and stored in a storage device (e.g., the storage device 130) of the medical system 100. The processing device 120 may obtain the mapping relationship between the first coordinate system associated with the medical device and the second coordinate system associated with the subject model from the storage device (e.g., the storage device 130).

Further, the processing device 120 may cause the terminal device to display the target image data. For example, the processing device 120 may cause the terminal device (e.g., the terminal device 140) associated with a user (e.g., a doctor) of the medical system 100 to display the target image data. In some embodiments, the processing device 120 may perform a rendering operation on the subject model based on the one or more historical scan regions and the one or more historical radiation doses. As used herein, a rendering operation refers to a process of generating a photorealistic or non-photorealistic image from a 2D or 3D model. In some embodiments, the one or more historical scan regions may be displayed in the subject model in different colors. For example, a first historical scan region (e.g., the head) may be displayed in the subject model in red color, a second historical scan region (e.g., the chest) may be displayed in the subject model in yellow color, and a third historical scan region (e.g., the abdomen) may be displayed in the subject model in green color.

In some embodiments, one or more colors of the one or more historical scan regions may correspond to the one or more historical radiation doses of the one or more historical scan regions. For example, if a first historical radiation dose of the first historical scan region is equal to or exceeds a corresponding dose threshold, the first historical scan region may be displayed in red. If a second historical radiation dose of the second historical scan region does not exceed a corresponding dose threshold, and the second historical scan region can receive additional radiation, the second historical scan region may be displayed in green. If a third historical radiation dose of the third historical scan region does not exceed a corresponding dose threshold, and the third historical scan region should not receive additional radiation, the third historical scan region may be displayed in yellow.

In some embodiments, different scan regions may correspond to different dose thresholds. In some embodiments, different scan regions may correspond to a same dose threshold. The dose threshold may be manually set by a user (e.g., a doctor) of the medical system 100, or by one or more components (e.g., the processing device 120) of the medical system 100 according to different situations. For example, a dose threshold corresponding to an accumulative radiation dose of an organ or tissue of a subject (e.g., a human body) in one year may be set as 50 mSv. As another example, a dose threshold corresponding to an accumulative radiation dose of a lens of the subject in one year may be set as 150 mSv.

In some embodiments, the processing device 120 (or the user) may determine whether an accumulative radiation dose corresponding to the one or more historical scans of a scan region of the subject exceeds a corresponding dose threshold based on the target image data. In response to determining that the accumulative radiation dose corresponding to the one or more historical scans of the scan region exceeds the dose threshold, the processing device 120 may generate a reminder. The reminder may be in the form of text, voice, a picture, a video, a haptic alert, or the like, or any combination thereof. In some embodiments, the reminder may indicate that the accumulative radiation dose of which portion(s) of the subject exceeds the dose threshold.

In some embodiments, the processing device 120 (or the user) may determine and/or adjust a scan protocol of the subject in a current scan based on the target image data and a scan region of the subject in the current scan. For example, the processing device 120 (or the user) may determine and/or adjust value(s) of at least one scanning parameter (e.g., a pitch, a tube voltage, a tube current) related to the current scan, so that after the current scan is performed on the scan region according to the adjusted value(s) of the at least one scanning parameter, an accumulative radiation dose corresponding to the one or more historical scans and the current scan of the scan region does not exceed the dose threshold, and an image generated based on scan data of the current scan can also satisfy the diagnosis requirements. In some embodiments, the processing device 120 may determine a recommended radiation dose of the scan region based on the accumulative radiation dose corresponding to the one or more historical scans of the scan region of the subject.

In some embodiments, the processing device 120 may determine a scan mode of the scan region based on the accumulative radiation dose corresponding to the one or more historical scans of the scan region of the subject. For example, if a historical CT scan is performed on the chest and the abdomen of the subject, and a scan region of the current scan is the chest of the subject. The processing device 120 may determine that a scan mode of the scan region as a focus-limited X-ray scan with an iris filter, so as to prevent regions close to the chest of the subject from receiving unnecessary radiation. As another example, a plain CT scan may be used to replace the spiral CT scan so as to prevent regions adjacent to the scan region from receiving unnecessary radiation.

In some embodiments, the processing device 120 may determine a position of at least one component (e.g., a collimator) of the medical device based on the accumulative radiation dose corresponding to the one or more historical scans of the scan region of the subject. For example, in response to determining that the accumulative radiation dose corresponding to the abdomen of the subject is equal to or close to a corresponding dose threshold (e.g., a difference between the accumulative radiation dose and the dose threshold is less than a difference threshold), the processing device 120 may control a radiation region of radiation beams (e.g., X-ray beams) emitted by a radiation source of the medical device (e.g., control the radiation beams not to irradiate the abdomen region of the subject) by adjusting the position of the collimator during the scan, so as to prevent the abdomen region from receiving unnecessary radiation.

In some embodiments, the processing device 120 and/or the user may adjust a boundary of a scan region based on the accumulative radiation dose corresponding to the one or more historical scans of the scan region of the subject.

In some embodiments, a medical device with collimation control technology may be used to prevent regions adjacent to the scan region from receiving unnecessary radiation. In some embodiments, a medical device with photon counting technology and/or iterative reconstruction technology may be used to adjust value(s) of at least one scanning parameter (e.g., a tube voltage, a tube current) related to the current scan, so that after the current scan is performed on the scan region according to the adjusted value(s) of the at least one scanning parameter, an accumulative radiation dose corresponding to the one or more historical scans and the current scan of the scan region does not exceed the dose threshold. In some embodiments, dose modulation technology may be used to control the radiation dose of the scan region of the subject. In some embodiments, a non-radioactive scan may be used to examine the subject, so as to prevent the subject from receiving additional radiation.

In some embodiments, if a first scan region is the head of the subject, and a second scan region is the chest of the subject in the current scan, and the accumulative radiation dose corresponding to the abdomen of the subject is equal to or close to a corresponding dose threshold, the processing device 120 may recommend that only the first scan region (i.e., the head) can be scanned in the current scan, and the second scan region (i.e., the chest) cannot be scanned in the current scan.

According to some embodiments of the present disclosure, the subject model, the one or more historical scan regions, and the one or more historical radiation doses may be displayed according to the one or more historical record modes on the terminal device associated with the user of the medical system 100. The user may view the historical scan information of the subject intuitively, and determine and/or adjust a scan protocol associated with the current scan of the subject based on the historical scan information of the subject, which may prevent the subject from receiving excessive radiation, and improve the safety of the subject during the current scan.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, process 600 may be used in a hospital information system (HIS), a laboratory information management system (LIS), a picture archiving and communication systems (PACS), a radiology information system (RIS), or the like, or any combination thereof.

In some embodiments, the processing device 120 may obtain scan information of the subject, as described in connection with operation 510. The processing device 120 may determine, based on the scan information, a distribution of a radiation dose in at least one dimension of a scan region of the subject, as described in connection with operation 510. The processing device 120 may provide the distribution of the radiation dose in a visualization model of at least a portion of the subject. For example, the processing device 120 may generate the subject model representing the at least a portion of the subject, as described in connection with operation 620. The processing device 120 may map the distribution of the radiation dose on the subject model, to generate the visualization model of the at least a portion of the subject, as described in connection with operation 630. The processing device 120 may cause the terminal device to display the visualization model, as described in connection with operation 630.

In some embodiments, the processing device 120 may obtain scan information (e.g., historical scan information) of a plurality of scans (e.g., a plurality of historical scans) of a subject, as described in connection with operation 610. The processing device 120 may determine, based on the scan information, a (current) distribution of an accumulative radiation dose corresponding to the plurality of scans in at least one dimension of the subject. The processing device 120 may provide the (current) distribution of the accumulative radiation dose in a visualization model of the subject. For example, the processing device 120 may generate the subject model representing the subject, as described in connection with operation 620. The processing device 120 may map the (current) distribution of the accumulative radiation dose on the subject model, to generate the visualization model of the subject, as described in connection with operation 630. The processing device 120 may cause the terminal device to display the visualization model, as described in connection with operation 630.

In some embodiments, the processing device 120 may determine the (current) distribution of the accumulative radiation dose corresponding to the plurality of scans based on the scan information (e.g., historical scan information) of the plurality of scans (e.g., the plurality of historical scans) of the subject and time information of the plurality of scans. For example, the processing device 120 may determine the (current) distribution of the accumulative radiation dose corresponding to the plurality of scans based on the radiation dose of each scan of the plurality of scans and a time weight corresponding to the each scan of the plurality of scans. The time weight corresponding to the scan may be determined based on a time of the scan (i.e., the time at which the scan is performed). For example, a longer time interval between a current time and the time of the scan may correspond to a smaller time weight corresponding to the scan. For illustration purposes, assuming that a radiation dose of a position of a subject is A in a first scan, a radiation dose of the position of the subject is B in a second scan, and a radiation dose of the position of the subject is C in a third scan, the time of the first scan is within one year from now, the time of the second scan is within 2-5 years from now, and the time of the third scan is five years ago from now, the processing device 120 may determine that a first time weight corresponding to the first scan is 1, a second time weight corresponding to the second scan is 0.4, and a third time weight corresponding to the third scan is 0. The processing device 120 may determine that an accumulative radiation dose corresponding to the plurality of scans (i.e., the first scan, the second scan, the third scan) on the position of the subject is (A*1+B*0.4+C*0). In some embodiments, a time weight corresponding to a scan may be determined based on a time weight curve. For example, the time weight curve may be determined by fitting a plurality of points (e.g., a first point indicating that a time weight is 0 when the time of the scan is 5 years ago from now, a second point indicating that a time weight is 0.4 when the time of the scan is within 2-5 years from now, a third point indicating that a time weight is 1 when the time of the scan is within one year from now).

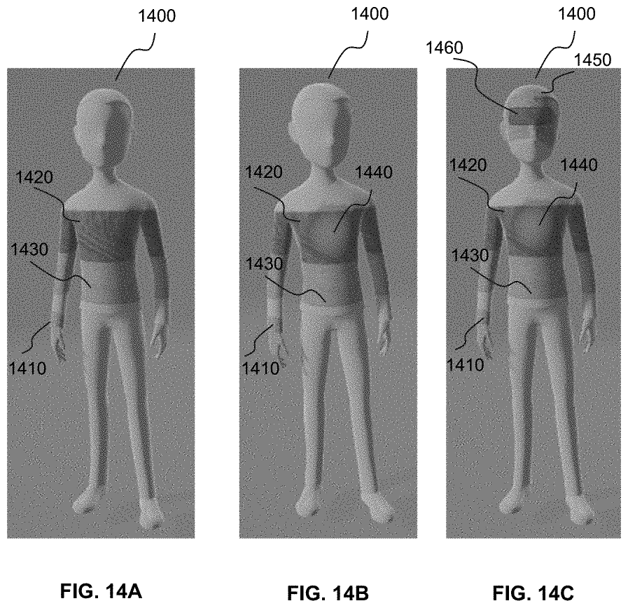

FIGS. 14A-14C are schematic diagrams illustrating exemplary historical scan processes of a subject according to some embodiments of the present disclosure.

In a first historical scan process, a CT scan is performed on a right hand region 1410, a first chest region 1420, and an abdomen region 1430 of the subject, as illustrated in FIG. 14A. In a second historical scan process, a focus-limited X-ray scan is performed on a second chest region 1440 of the subject, as illustrated in FIG. 14B. In a third historical scan process, a CT scan with a reference region (e.g., an eye region 1460) protection is performed on a head region 1450 of the subject, as illustrated in FIG. 14C. The historical scan regions (e.g., the right hand region 1410, the first chest region 1420, the abdomen region 1430, the second chest region 1440, the head region 1450, the eye region 1460) are displayed in a subject model 1400 in different colors. The colors of the historical scan regions correspond to historical radiation doses of the historical scan regions.

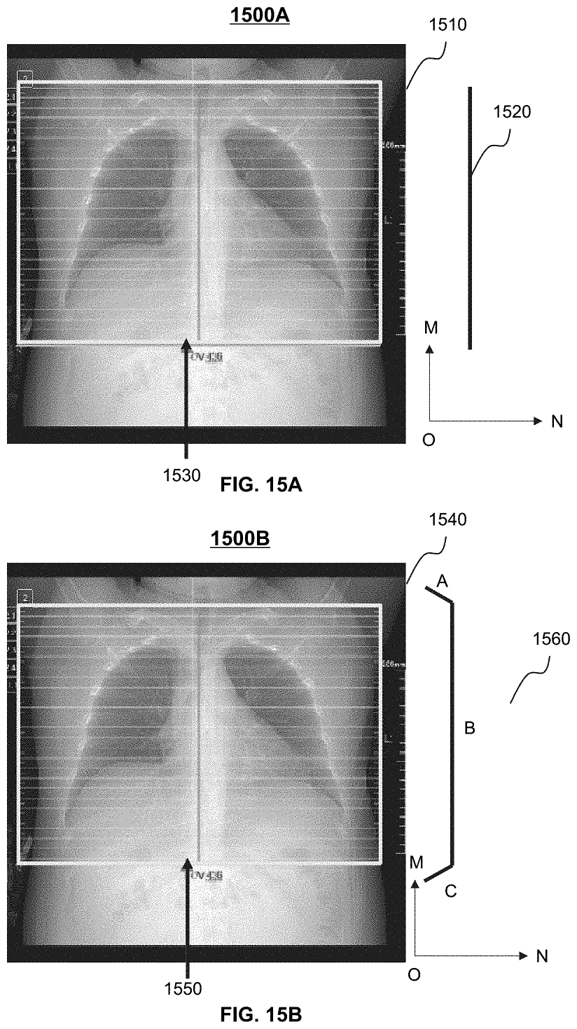

FIG. 15A is a schematic diagram illustrating an exemplary user interface according to some embodiments of the present disclosure. As illustrated in FIG. 15A, a user interface 1500A may include a topogram image 1510 of a subject and a radiation dose curve 1520. A scan mode of a scan region 1530 (e.g., a lung region) of the subject may be a plain CT scan. An M-axis of a radiation dose curve (e.g., the radiation dose curve 1520) may represent a position (e.g., the Z-axis coordinate of the position) of a scan region (e.g., the scan region 1530) along a scan axis direction (e.g., the Z-axis direction as illustrated in FIG. 1) of a medical device (e.g., a CT device). An N-axis of a radiation dose curve (e.g., the radiation dose curve 1520) may represent a radiation dose. The radiation dose curve 1520 may reflect radiation doses corresponding to a plurality of layers of the scan region 1530 of the subject. As illustrated in FIG. 15A, the radiation dose curve 1520 may be a straight line representing the radiation doses corresponding to the plurality of layers of the scan region 1530 maintains a target radiation dose.

FIG. 15B is a schematic diagram illustrating an exemplary user interface according to some embodiments of the present disclosure. As illustrated in FIG. 15B, a user interface 1500B may include a topogram image 1540 of a subject and a radiation dose curve 1560. A scan mode of a scan region 1550 (e.g., a lung region) of the subject may be a spiral CT scan. The radiation dose curve 1560 may reflect radiation doses corresponding to a plurality of layers of the scan region 1550 of the subject. The radiation dose curve 1560 may include a first section A, a second section B, and a third section C, as described in connection with FIG. 7.

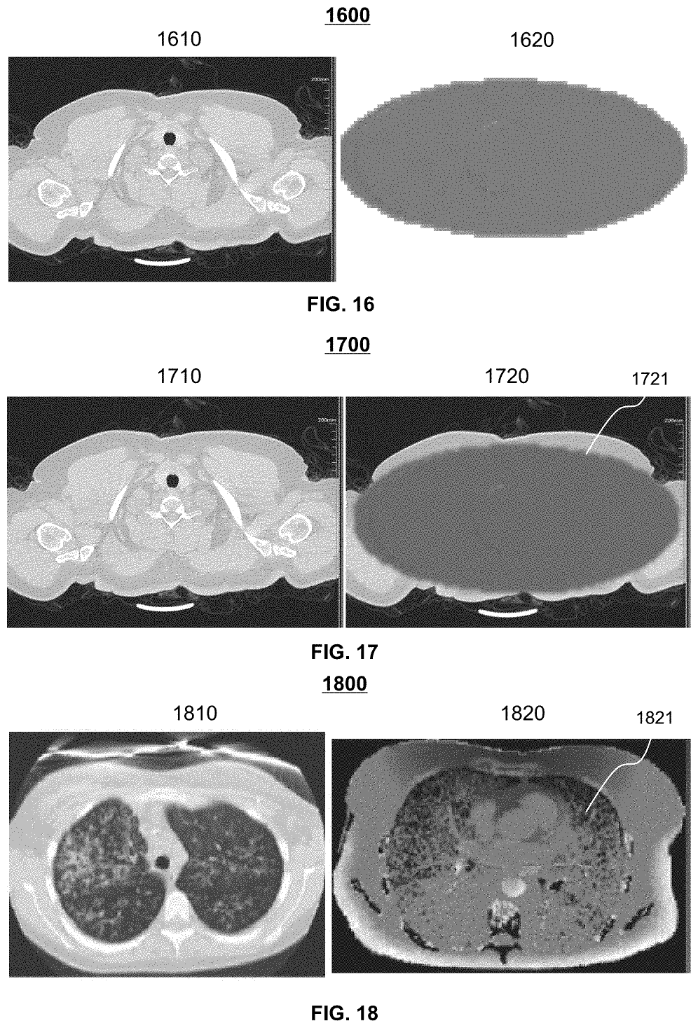

FIG. 16 is a schematic diagram illustrating an exemplary user interface according to some embodiments of the present disclosure. As illustrated in FIG. 16, a user interface 1600 may include a topogram image 1610 of a subject and a model 1620. The topogram image 1610 may correspond to a cross section of the subject. A scan mode of a scan region (e.g., a spine region) of the subject may be a CT scan. In some embodiments, a CT device may scan the scan region along a scan axis direction. The scan axis direction and an axis direction of the subject (e.g., the Z-axis direction as illustrated in FIG. 1) may have an angle. The axis direction of the subject may be perpendicular to the cross section of the subject. The model 1620 may reflect radiation doses corresponding to a plurality of layers of the scan region of the subject. In some embodiments, an angle-based dose modulation and organ protection techniques may cause dose changes in at least one dimension (e.g., X-axis and Y-axis directions as illustrated in FIG. 1) of the scan region of the subject. The model 1620 may reflect a distribution of a radiation dose in the scan region of the subject intuitively.

FIG. 17 is a schematic diagram illustrating an exemplary user interface according to some embodiments of the present disclosure. As illustrated in FIG. 17, a user interface 1700 may include a topogram image 1710 of a subject and target image data 1720. In the target image data 1720, a representation 1721 of a model may be superimposed on a representation of a scan region of the subject. By superimposing the representation 1721 of the model on the representation of the scan region of the subject, the distribution of the radiation dose of the scan region of the subject may be represented intuitively.

FIG. 18 is a schematic diagram illustrating an exemplary user interface according to some embodiments of the present disclosure. As illustrated in FIG. 18, a user interface 1800 may include a topogram image 1810 of a subject and target image data 1820. In the target image data 1820, a representation 1821 of a model may be superimposed on a representation of a scan region of the subject. In some embodiments, the representation 1821 of the model may be transparent or semi-transparent. Accordingly, in the target image data 1820, the distribution of the radiation dose of the scan region of the subject may be represented intuitively.

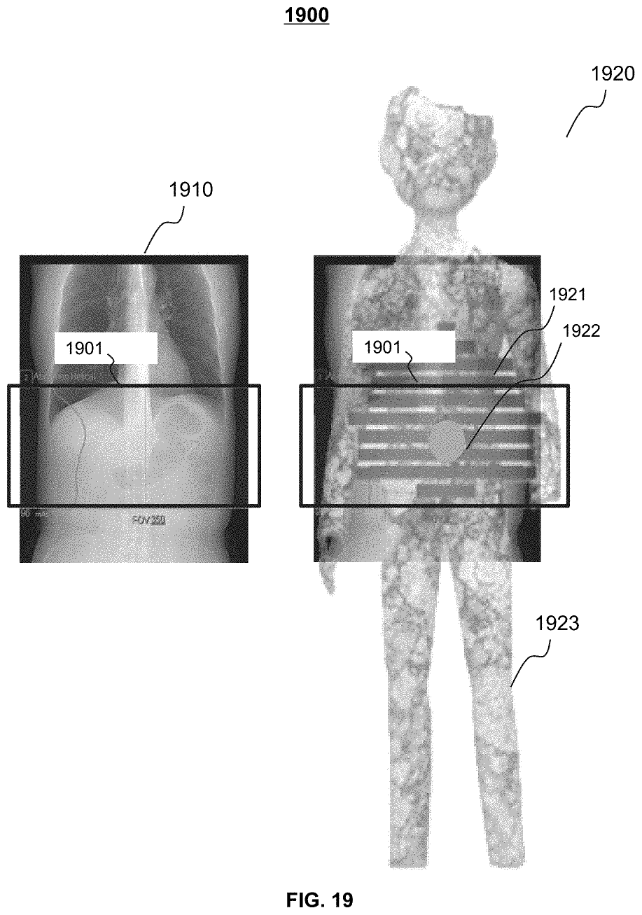

FIG. 19 is a schematic diagram illustrating an exemplary user interface according to some embodiments of the present disclosure. As illustrated in FIG. 19, a user interface 1900 may include a topogram image 1910 of a subject and target image data 1920. In the target image data 1920, a representation 1921 of a model including a plurality of elliptical cylinders with a curved surface 1922 may be superimposed on a representation 1923 of a subject model. The model may reflect radiation doses corresponding to a plurality of layers of a scan region 1901 of the subject.

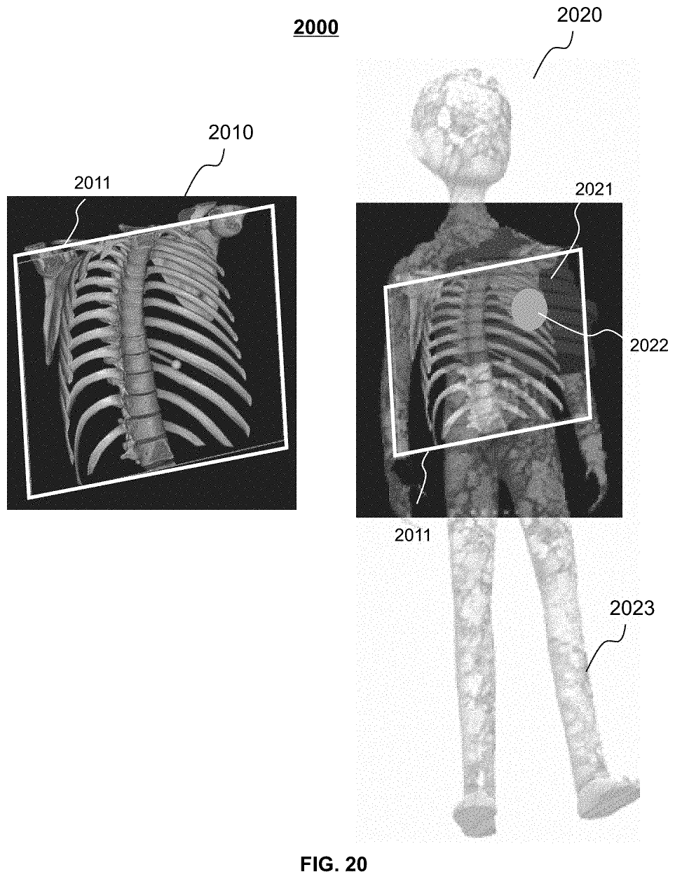

FIG. 20 is a schematic diagram illustrating an exemplary user interface according to some embodiments of the present disclosure. As illustrated in FIG. 20, a user interface 2000 may include a topogram image 2010 of a subject and target image data 2020. In the target image data 2020, a representation 2021 of a model including a plurality of elliptical cylinders with a curved surface 2022 may be superimposed on a representation 2023 of a subject model. The model may reflect radiation doses corresponding to a plurality of layers of a scan region 2011 of the subject. In some embodiments, the medical system 100 may adjust a display angle of the topogram image 2010 (or the target image data 2020) automatically. In some embodiments, a user (e.g., a doctor) of the medical system 100 may adjust the display angle of the topogram image 2010 (or the target image data 2020) manually.

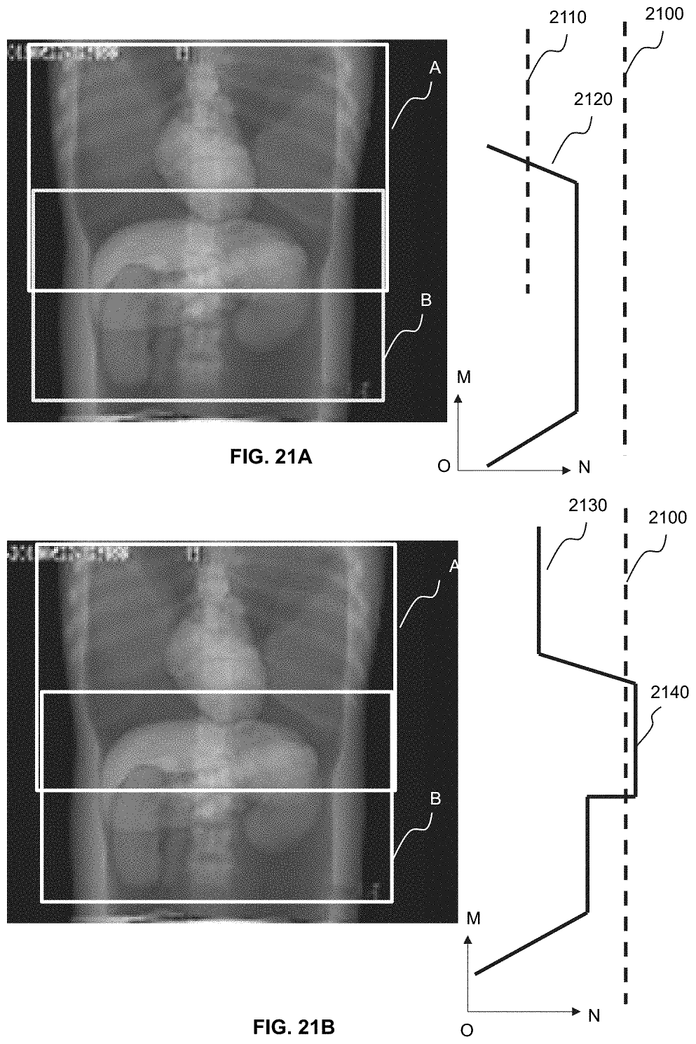

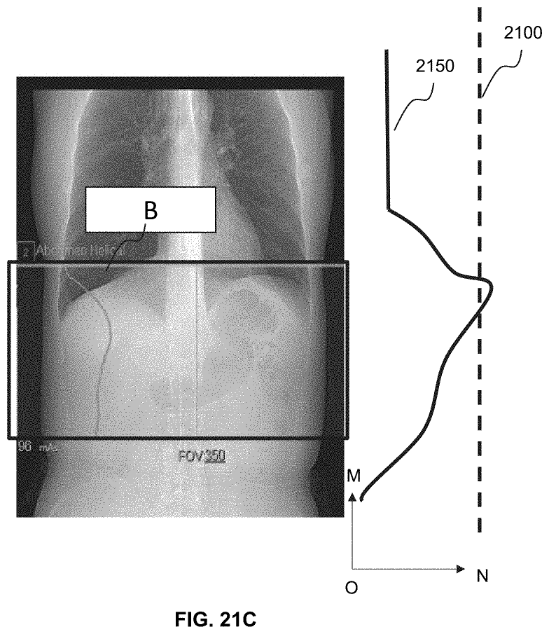

FIGS. 21A-21C are schematic diagrams illustrating exemplary scan processes of a subject according to some embodiments of the present disclosure.

As illustrated in FIG. 21A, in a first scan process, a plain CT scan is performed on a first region A (e.g., a lung region) of a subject, and a first radiation dose curve 2110 is generated based on scan information of the first scan process. A radiation dose corresponding to the first region A is below a dose threshold 2100 in the first scan process. In a second scan process, a spiral CT scan is performed on a second region B (e.g., an abdomen region) of the subject, and a second radiation dose curve 2120 is generated based on scan information of the second scan process. A radiation dose corresponding to the second region B is below the dose threshold 2100 in the second scan process.

As illustrated in FIG. 21B, the first radiation dose curve 2110 and the second radiation dose curve 2120 are combined into (e.g., accumulated to obtain) a third radiation dose curve 2130. A radiation dose of each point on the third radiation dose curve 2130 may be a sum of a radiation dose of a corresponding point on the first radiation dose curve 2110 and a radiation dose of a corresponding point on the second radiation dose curve 2120. A radiation dose corresponding to a section 2140 on the third radiation dose curve 2130 is greater than the dose threshold 2100. The section 2140 on the third radiation dose curve 2130 may correspond to an overlapping region of the first region A and the second region B of the subject. A user may adjust the radiation dose of the overlapping region (e.g., the lung region) of the first region A and the second region B by adjusting a scan protocol of the first scan process and/or a scan protocol of the second scan process, in order to avoid the accumulation of excessive radiation dose in the overlapping region. For example, the user may adjust the radiation dose of the overlapping region according to one or more dose modulation algorithms (e.g., a topogram image-based dose modulation algorithm). As illustrated in FIG. 21C, after the user adjusts the radiation dose of the overlapping region in the second scan process, a fourth radiation dose curve 2150 is generated, and the radiation dose of the overlapping region is reduced.

In some embodiments, before the first scan process and/or the second scan process, the first radiation dose curve 2110, the second radiation dose curve 2120, and the third radiation dose curve 2130 may be simulated. The processing device 120 and/or the user may determine whether an estimated radiation dose of at least one portion of the first scan region A and the second scan region B exceeds the dose threshold 2100 based on the first radiation dose curve 2110, the second radiation dose curve 2120, and/or the third radiation dose curve 2130. In response to determining that an estimated radiation dose of at least one portion of the first scan region A and the second scan region B exceeds the dose threshold 2100, the processing device 120 may generate a reminder. The processing device 120 (or the user) may determine and/or adjust a scan protocol of the first scan process and/or a scan protocol of the second scan process based on the reminder, as described elsewhere in the present disclosure.

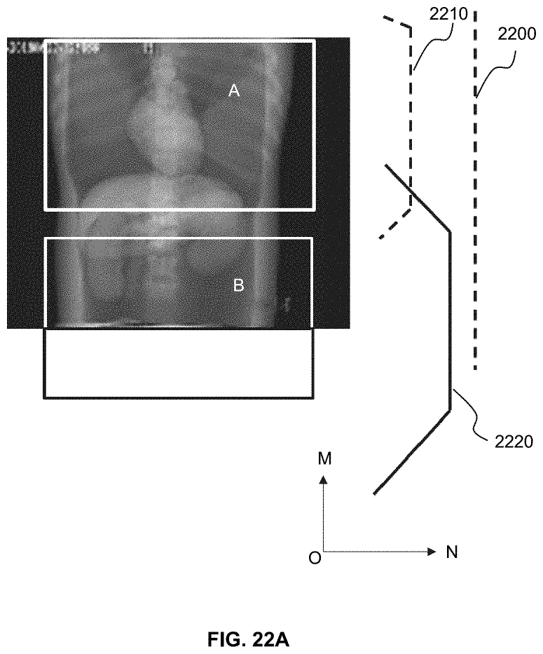

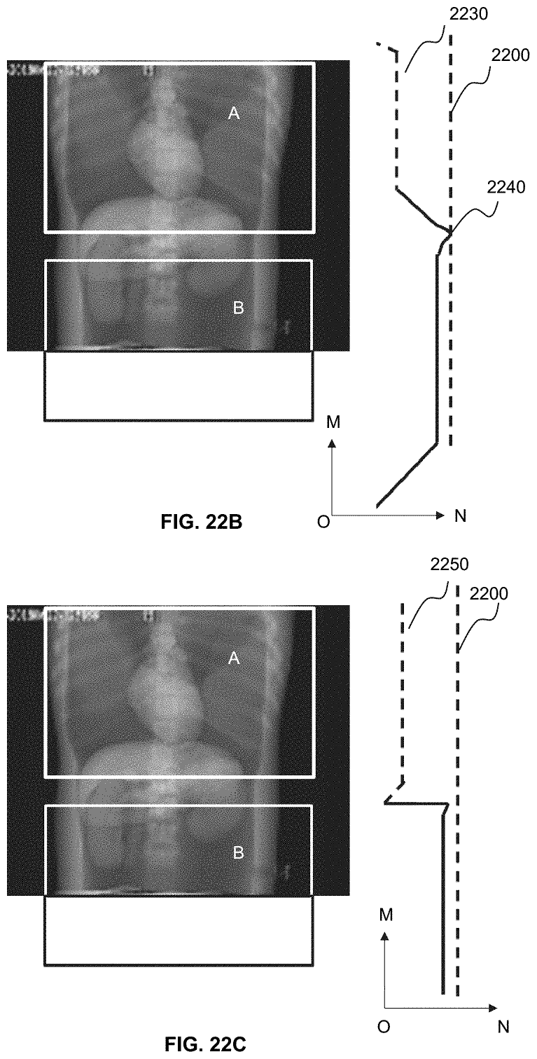

FIGS. 22A-22C are schematic diagrams illustrating exemplary scan processes of a subject according to some embodiments of the present disclosure.

As illustrated in FIG. 22A, in a first scan process, a spiral CT scan is performed on a first region A (e.g., a lung region) of a subject, and a first radiation dose curve 2210 is generated based on scan information of the first scan process. A radiation dose corresponding to the first region A is below a dose threshold 2200 in the first scan process. In a second scan process, a spiral CT scan is performed on a second region B (e.g., an abdomen region) of the subject, and a second radiation dose curve 2220 is generated based on scan information of the second scan process. A radiation dose corresponding to the second region B is below the dose threshold 2200 in the second scan process.

As illustrated in FIG. 22B, the first radiation dose curve 2210 and the second radiation dose curve 2220 are combined into (e.g., accumulated to obtain) a third radiation dose curve 2230. A radiation dose of each point on the third radiation dose curve 2230 may be a sum of a radiation dose of a corresponding point on the first radiation dose curve 2210 and a radiation dose of a corresponding point on the second radiation dose curve 2220. A radiation dose corresponding to a section 2240 on the third radiation dose curve 2230 is close to the dose threshold 2200. The section 2240 on the third radiation dose curve 2130 may correspond to an overlapping region of the first region A and the second region B of the subject. A user may adjust the radiation dose of the overlapping region of the first region A and the second region B by adjusting a scan protocol of the first scan process and/or a scan protocol of the second scan process of the subject, in order to avoid the accumulation of excessive radiation dose in the overlapping region. For example, a san mode, and/or a value of at least one scanning parameter (e.g., a collimation width, a pitch, a thickness of a scan layer) of the first scan and/or the second scan may be adjusted. As illustrated in FIG. 22C, after the user adjusts the scan mode of second scan process from the spiral CT scan mode to a plain CT scan mode, a fourth radiation dose curve 2250 is generated, and the radiation dose of the overlapping region is reduced.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

What is claimed is:

1. A method for radiation dose management, which is implemented on a computing device including at least one processor and at least one storage device, comprising:
obtaining scan information of a subject, wherein the scan information includes a scan mode, a scan region of the subject, and a radiation dose of the subject;
determining a record mode based on the scan mode; and
recording the scan region and the radiation dose based on the record mode.

2. The method of claim 1, wherein the recording the scan region and the radiation dose based on the record mode comprises:
determining a radiation dose corresponding to each position of a plurality of positions distributed along at least one direction of the scan region based on the radiation dose of the subject; and
recording the radiation dose corresponding to the each position of the plurality of positions of the scan region based on the record mode.

3. The method of claim 1, wherein the scan mode includes a spiral CT scan, and the determining a record mode based on the scan mode comprises:
determining the record mode as a radiation dose curve based on the spiral CT scan.

4. The method of claim 3, wherein the recording the scan region and the radiation dose based on the record mode comprises:
determining a radiation dose of each layer of a plurality of layers of the scan region;
determining the radiation dose curve based on radiation doses of the plurality of layers of the scan region; and
recording the radiation doses corresponding to the plurality of layers of the scan region using the radiation dose curve.

5. The method of claim 1, wherein the scan mode includes a CT scan, and the determining a record mode based on the scan mode comprises:
determining the record mode as a first model including a plurality of elliptical cylinders based on the CT scan, the first model being configured to reflect radiation doses corresponding to a plurality of layers of the scan region.

6. The method of claim 5, wherein the scan information further includes a thickness of each layer of the plurality of layers of the scan region, and the recording the scan region and the radiation dose based on the record mode comprises:
determining a radiation dose of the each layer of the plurality of layers of the scan region;
generating the first model based on the thickness of the each layer of the plurality of layers of the scan region, and the radiation dose of the each layer of the plurality of layers of the scan region, wherein a height of each elliptical cylinder of the plurality of elliptical cylinders corresponds to a thickness of a corresponding layer of the plurality of layers of the scan region, and a cross-sectional area of the each elliptical cylinder corresponds to a radiation dose of the corresponding layer; and
recording radiation doses corresponding to the plurality of layers of the scan region using the first model.

7. The method of claim 1, wherein the scan mode includes a CT scan, the scan region includes a reference region, and the determining a record mode based on the scan mode comprises:
determining the record mode as a second model based on the CT scan and the reference region, wherein the second model includes a cylinder with a first curved surface, and the first curved surface corresponds to the reference region.

8. The method of claim 7, wherein the scan information further includes a radiation dose of the reference region, and the recording the scan region and the radiation dose based on the record mode comprises:
generating the cylinder based on the scan region and the radiation dose of the subject, wherein a longitudinal axis of the cylinder is perpendicular to a cross section of the subject;
generating the second model by forming, based on the radiation dose of the reference region, the first curved surface on the cylinder; and
recording radiation doses corresponding to a plurality of positions of the reference region using the second model.

9. The method of claim 1, wherein the scan mode includes a CT scan, the scan region includes a reference region, and the determining a record mode based on the scan mode comprises:
determining the record mode as a third model based on the CT scan and the reference region, wherein the third model includes a plurality of elliptical cylinders with a second curved surface, the second curved surface corresponds to the reference region, and the third model is configured to reflect radiation doses corresponding to a plurality of layers of the scan region.

10. The method of claim 9, wherein the scan information further includes a thickness of each layer of the plurality of layers of the scan region, and a radiation dose of the reference region, and the recording the scan region and the radiation dose based on the record mode comprises:
determining a radiation dose of the each layer of the plurality of layers of the scan region;
generating the plurality of elliptical cylinders based on the thickness of the each layer of the plurality of layers of the scan region, and the radiation dose of the each layer of the plurality of layers of the scan region, wherein a longitudinal axis of the each elliptical cylinder of the plurality of elliptical cylinders is located on a same straight line, the longitudinal axis of the each elliptical cylinder of the plurality of elliptical cylinders is perpendicular to a cross section of the subject, a height of the each elliptical cylinder of the plurality of elliptical cylinders corresponds to a thickness of a corresponding layer of the plurality of layers of the scan region, and a cross-sectional area of the each elliptical cylinder corresponds to a radiation dose of the corresponding layer;
generating the third model by forming, based on the radiation dose of the reference region, the second curved surface on the plurality of elliptical cylinders; and
recording radiation doses corresponding to the plurality of layers of the scan region and a plurality of positions of the reference region using the third model.

11. The method of claim 1, wherein the scan mode includes a beam limited scan mode, and the determining a record mode based on the scan mode comprises:
determining the record mode as a fourth model based on the beam limited scan mode, wherein the fourth model includes a frustum.

12. The method of claim 11, wherein the scan information further includes a shape of a beam limiter of a medical device, and the recording the scan region and the radiation dose based on the record mode comprises:
generating the fourth model based on the shape of the beam limiter and the radiation dose of the subject, wherein an axis of the frustum is perpendicular to a coronal plane of the subject, a shape of a surface of the frustum corresponds to the shape of the beam limiter, and a height of the frustum corresponds to the radiation dose of the subject; and
recording the radiation dose of the subject using the fourth model.

13. The method of claim 1, further comprising:
obtaining one or more historical scan regions of the subject, one or more historical radiation doses of the subject, and one or more historical record modes;
generating a subject model representing the subject; and
causing a terminal device to display the subject model, the one or more historical scan regions and the one or more historical radiation doses according to the one or more historical record modes.

14. The method of claim 13, wherein the causing a terminal device to display the subject model, the one or more historical scan regions, and the one or more historical radiation doses according to the one or more historical record modes com prises:
generating target image data by mapping the one or more historical scan regions and the one or more historical radiation doses on the subject model according to the one or more historical record modes; and
causing the terminal device to display the target image data.

15. The method of claim 14, wherein the one or more historical scan regions are displayed in the subject model in different colors, or one or more colors of the one or more historical scan regions correspond to the one or more historical radiation doses of the one or more historical scan regions.

16. The method of claim 1, wherein the recording the scan region and the radiation dose based on the record mode comprises:
generating a model based on the record mode; and
visualizing the scan region and the radiation dose using the model.

17. The method of claim 16, wherein the scan information includes historical scan information of at least one historical scan of the subject, and the visualizing the scan region and the radiation dose using the model comprises:
displaying a current distribution of an accumulative radiation dose corresponding to the at least one historical scan of the subject in the model.

18. The method of claim 17, wherein the scan information further includes current scan information of a current scan of the subject, and the visualizing the scan region and the radiation dose using the model comprises:
displaying an estimated distribution of an accumulative radiation dose corresponding to the at least one historical scan of the subject and the current scan of the subject in the model.

19. A method for radiation dose management, which is implemented on a computing device including at least one processor and at least one storage device, comprising:
obtaining scan information of a subject;
determining, based on the scan information, a distribution of a radiation dose in at least one dimension of a scan region of the subject; and
displaying the distribution of the radiation dose in a visualization model of at least a portion of the subject.

20. A method for radiation dose management, which is implemented on a computing device including at least one processor and at least one storage device, comprising:
obtaining historical scan information of a plurality of historical scans of a subject;
determining, based on the historical scan information, a current distribution of an accumulative radiation dose corresponding to the plurality of historical scans in at least one dimension of the subject; and
displaying the current distribution of the accumulative radiation dose in a visualization model of the subject.
